(19) United States
(12) Patent Application Publication
(10) Pub. No.: US 2003/0158396 A1
(43) Pub. Date: Aug. 21, 2003

(54) Identification of polynucleotides encoding novel helicobacter polypeptides in the helicobacter genome

(76) Inventors: Harold Kleanthous, Newtonville, MA (US); Amal Al-Garawi, Brookline, MA (US); Charles Miller, Medford, MA (US); Jean-Francois Tomb, Baltimore, MD (US); Raymond P. Oomen, Ontario (CA)

Correspondence Address: CLARK & ELBING LLP, 101 FEDERAL STREET, BOSTON, MA, 02110, US

(21) Appl. No.: 09/882,227

(22) Filed: Jun. 15, 2001

Related U.S. Application Data
(63) Continuation of application No. 08/902,615, filed on Jul. 29, 1997, now abandoned.

(51) Int. Cl. C07H 21/02; C07H 21/04

(52) U.S. Cl. 536/23.1

(57) ABSTRACT

The invention provides Helicobacter polypeptides that can be used in vaccination methods for preventing or treating Helicobacter infection, and polynucleotides that encode these polypeptides.

### PRIORITY INFORMATION

This application is a continuation of, and claims priority from, U.S. Ser. No. 08/902,615, filed on Jul. 29, 1997, which is incorporated by reference herein in its entirety.

### FIELD OF THE INVENTION

The invention relates to Helicobacter antigens and corresponding polynucleotide molecules that can be used in methods to prevent or treat Helicobacter infection in mammals, such as humans.

### BACKGROUND OF THE INVENTION

Helicobacter is a genus of spiral, gram-negative bacteria that colonize the gastrointestinal tracts of mammals. Several species colonize the stomach, most notably H. pylori, H. heilmanii, H. felis, and H. mustelae. Although H. pylori is the species most commonly associated with human infection, H. heilmanii and H. felis have also been isolated from humans, but at lower frequencies than H. pylori. Helicobacter infects over 50% of adult populations in developed countries and nearly 100% in developing countries and some Pacific rim countries, making it one of the most prevalent infections worldwide.

Helicobacter is routinely recovered from gastric biopsies of humans with histological evidence of gastritis and peptic ulceration. Indeed, H. pylori is now recognized as an important pathogen of humans, in that the chronic gastritis it causes is a risk factor for the development of peptic ulcer diseases and gastric carcinoma. It is thus highly desirable to develop safe and effective vaccines for preventing and treating Helicobacter infection.

A number of Helicobacter antigens have been characterized or isolated. These include urease, which is composed of two structural subunits of approximately 30 and 67 kDa (Hu et al., Infect. Immun. 58:992, 1990; Dunn et al., J. Biol. Chem. 265:9464, 1990; Evans et al., Microbial Pathogenesis 10:15, 1991; Labigne et al., J. Bact., 173:1920, 1991); the 87 kDa vacuolar cytotoxin (VacA) (Cover et al., J. Biol. Chem. 267:10570, 1992; Phadnis et al., Infect. Immun. 62:1557, 1994; WO 93/18150); a 128 kDa immunodominant antigen associated with the cytotoxin (CagA, also called TagA; WO 93/18150; U.S. Pat. No. 5,403,924); 13 and 58 kDa heat shock proteins HspA and HspB (Suerbaum et al., Mol. Microbiol. 14:959, 1994; WO 93/18150); a 54 kDa catalase (Hazell et al., J. Gen. Microbiol.137:57, 1991); a 15 kDa histidine-rich protein (Hpn) (Gilbert et al., Infect. Immun. 63:2682, 1995); a 20 kDa membrane-associated lipoprotein (Kostrcynska et al., J. Bact. 176:5938, 1994); a 30 kDa outer membrane protein (Bolin et 15 al., J. Clin. Microbiol. 33:381, 1995); a lactoferrin receptor (FR 2,724,936); and several porins, designated HopA, HopB, HopC, HopD, and HopE, which have molecular weights of 48-67 kDa (Exner et al., Infect. Immun. 63:1567, 1995; Doig et al., J. Bact. 177:5447, 1995). Some of these proteins have been proposed as potential vaccine antigens. In particular, urease is believed to be a vaccine candidate (WO 94/9823; WO 95/22987; WO 95/3824; Michetti et al., Gastroenterology 107:1002, 1994). Nevertheless, it is thought that several antigens may ultimately be necessary in a vaccine.

### SUMMARY OF THE INVENTION

The invention provides polynucleotide molecules that encode Helicobacter polypeptides, designated GHPO7 (SEQ ID NO: 2), GHPO8 (SEQ ID NO: 4), GHPO9 (SEQ ID NO: 6), GHPO10 (SEQ ID NO: 8), GHPO12 (SEQ ID NO: 10), GHPO25 (SEQ ID NO: 12), GHPO27 (SEQ ID NO: 14), GHPO29 (SEQ ID NO: 16), GHPO30 (SEQ ID NO: 18), GHPO37 (SEQ ID NO: 20), GHPO49 (SEQ ID NO: 22), GHPO51 (SEQ ID NO: 24), GHPO54 (SEQ ID NO: 26), GHPO65 (SEQ ID NO: 28), GHPO66 (SEQ ID NO: 30), GHPO68 (SEQ ID NO: 32), GHPO70 (SEQ ID NO: 34), GHPO77 (SEQ ID NO: 36), GHPO83 (SEQ ID NO: 38), GHPO85 (SEQ ID NO: 40), GHPO87 (SEQ ID NO: 42), GHPO91 (SEQ ID NO: 44), GHPO92 (SEQ ID NO: 46), GHPO96 (SEQ ID NO: 48), GHPO97 (SEQ ID NO: 50), GHPO111 (SEQ ID NO: 52), GHPO115 (SEQ ID NO: 54), GHPO117 (SEQ ID NO: 56), GHPO123 (SEQ ID NO: 58), GHPO124 (SEQ ID NO: 60), GHPO126 (SEQ ID NO: 62), GHPO127 (SEQ ID NO: 64), GHPO128 (SEQ ID NO: 66), GHPO131 (SEQ ID NO: 68), GHPO133 (SEQ ID NO: 70), GHPO140 (SEQ ID NO: 72), GHPO141 (SEQ ID NO: 74), GHPO145 (SEQ ID NO: 76), GHPO147 (SEQ ID NO: 78), GHPO166 (SEQ ID NO: 80), GHPO181 (SEQ ID NO: 82), GHPO187 (SEQ ID NO: 84), GHPO188 (SEQ ID NO: 86), GHPO192 (SEQ ID NO: 88), GHPO202 (SEQ ID NO: 90), GHPO204 (SEQ ID NO: 92), GHPO205 (SEQ ID NO: 94), GHPO212 (SEQ ID NO: 96), GHPO218 (SEQ ID NO: 98), GHPO226 (SEQ ID NO:l00), GHPO231 (SEQ ID NO: 102), GHPO236 (SEQ ID NO: 104), GHPO239 (SEQ ID NO: 106), GHPO245 (SEQ ID NO: 108), GHPO246 (SEQ ID NO: 110), GHPO248 (SEQ ID NO: 112), GHPO253 (SEQ ID NO: 114), GHPO265 (SEQ ID NO: 116), GHPO266 (SEQ ID NO: 118), GHPO271 (SEQ ID NO: 120), GHPO272 (SEQ ID NO: 122), GHPO286 (SEQ ID NO: 124), GHPO291 (SEQ ID NO: 126), GHPO292 (SEQ ID NO: 128), GHPO297 (SEQ ID NO: 130), GHPO304 (SEQ ID NO: 132), GHPO307 (SEQ ID NO: 134), GHPO324 (SEQ ID NO: 136), GHPO326 (SEQ ID NO: 138), GHPO331 (SEQ ID NO: 140), GHPO343 (SEQ ID NO: 142), GHPO345 (SEQ ID NO: 144), GHPO346 (SEQ ID NO: 146), GHPO352 (SEQ ID NO: 148), GHPO355 (SEQ ID NO: 150), GHPO363 (SEQ ID NO: 152), GHPO369 (SEQ ID NO: 154), GHPO376 (SEQ ID NO: 156), GHPO378 (SEQ ID NO: 158), GHPO388 (SEQ ID NO: 160), GHPO396 (SEQ ID NO: 162), GHPO403 (SEQ ID NO: 164), GHPO410 (SEQ ID NO: 166), GHPO415 (SEQ ID NO: 168), GHPO421 (SEQ ID NO: 170), GHPO439 (SEQ ID NO: 172), GHPO441 (SEQ ID NO: 174), GHPO443 (SEQ ID NO: 176), GHPO453 (SEQ ID NO: 178), GHPO455 (SEQ ID NO: 180), GHPO464 (SEQ ID NO: 182), GHPO467 (SEQ ID NO: 184), GHPO468 (SEQ ID NO: 186), GHPO470 (SEQ ID NO: 188), GHPO486 (SEQ ID NO: 190), GHPO487 (SEQ ID NO: 192), GHPO488 (SEQ ID NO: 194), GHPO489 (SEQ ID NO: 196), GHPO498 (SEQ ID NO: 198), GHPO501 (SEQ ID NO: 200), GHPO504 (SEQ ID NO: 202), GHPO512 (SEQ ID NO: 204), GHPO517 (SEQ ID NO: 206), GHPO520 (SEQ ID NO: 208), GHPO528 (SEQ ID NO: 210), GHPO530 (SEQ ID NO: 212), GHPO532 (SEQ ID NO: 214), GHPO548 (SEQ ID NO: 216), GHPO561 (SEQ ID NO: 218), GHPO564 (SEQ ID NO: 220), GHPO572 (SEQ ID NO: 222), GHPO573 (SEQ ID NO: 224), GHPO574 (SEQ ID NO: 226), GHPO577 (SEQ ID NO: 228), GHPO579 (SEQ ID NO: 230), GHPO583 (SEQ ID NO: 232), GHPO588 (SEQ ID NO: 234), GHPO593 (SEQ ID NO: 236), GHPO597 (SEQ ID NO: 238), GHPO598 (SEQ ID NO: 240), GHPO604 (SEQ ID NO: 242), GHPO606 (SEQ ID NO: 244), GHPO611 (SEQ ID NO: 246), GHPO612 (SEQ ID NO: 248), GHPO615 (SEQ ID NO: 250), GHPO632 (SEQ ID NO: 252), GHPO633 (SEQ ID NO: 254), GHPO637 (SEQ ID NO: 256), GHPO651 (SEQ ID NO: 258), GHPO663 (SEQ ID NO: 260), GHPO686 (SEQ ID NO: 262), GHPO693 (SEQ ID NO: 264), GHPO698 (SEQ ID NO: 266), GHPO703 (SEQ ID NO: 268), GHPO704 (SEQ ID NO: 270), GHPO705 (SEQ ID NO: 272), GHPO707 (SEQ ID NO: 274), GHPO721 (SEQ ID NO: 276), GHPO727 (SEQ ID NO: 278), GHPO728 (SEQ ID NO: 280), GHPO733 (SEQ ID NO: 282), GHPO758 (SEQ ID NO: 284), GHPO763 (SEQ ID NO: 286), GHPO771 (SEQ ID NO: 288), GHPO774 (SEQ ID NO: 290), GHPO776 (SEQ ID NO: 292), GHPO783 (SEQ ID NO: 294), GHPO800 (SEQ ID NO: 296), GHPO806 (SEQ ID NO: 298), GHPO807 (SEQ ID NO: 300), GHPO808 (SEQ ID NO: 302), GHPO809 (SEQ ID NO: 304), GHPO811 (SEQ ID NO: 306), GHPO815 (SEQ ID NO: 308), GHPO819 (SEQ ID NO: 310), GHPO841 (SEQ ID NO: 312), GHPO843 (SEQ ID NO: 314), GHPO846 (SEQ ID NO: 316), GHPO875 (SEQ ID NO: 318), GHPO892 (SEQ ID NO: 320), GHPO902 (SEQ ID NO: 322), GHPO904 (SEQ ID NO: 324), GHPO906 (SEQ ID NO: 326), GHPO908 (SEQ ID NO: 328), GHPO921 (SEQ ID NO: 330), GHPO923 (SEQ ID NO: 332), GHPO926 (SEQ ID NO: 334), GHPO933 (SEQ ID NO: 336), GHPO939 (SEQ ID NO: 338), GHPO940 (SEQ ID NO: 340), GHPO943 (SEQ ID NO: 342), GHPO951 (SEQ ID NO: 344), GHPO961 (SEQ ID NO: 346), GHPO965 (SEQ ID NO: 348), GHPO990 (SEQ ID NO: 350), GHPO991 (SEQ ID NO: 352), GHPO998 (SEQ ID NO: 354), GHPO1001 (SEQ ID NO: 356), GHPO1005 (SEQ ID NO: 358), GHPO1033 (SEQ ID NO: 360), GHPO1039 (SEQ ID NO: 362), GHPO1041 (SEQ ID NO: 364), GHPO1043 (SEQ ID NO: 366), GHPO1044 (SEQ ID NO: 368), GHPO1051 (SEQ ID NO: 370), GHPO1058 (SEQ ID NO: 372), GHPO1060 (SEQ ID NO: 374), GHPO1075 (SEQ ID NO: 376), GHPO1077 (SEQ ID NO: 378), GHPO1082 (SEQ ID NO: 380), GHPO1083 (SEQ ID NO: 382), GHPO1086 (SEQ ID NO: 384), GHPO1087 (SEQ ID NO: 386), GHPO1090 (SEQ ID NO: 388), GHPO1097 (SEQ ID NO: 390), GHPO1098 (SEQ ID NO: 392), GHPO1103 (SEQ ID NO: 394), GHPO1113 (SEQ II) NO: 396), GHPO1116 (SEQ ID NO: 398), GHPO1123 (SEQ ID NO: 400), GHPO1125 (SEQ ID NO: 402), GHPO1129 (SEQ ID NO: 404), GHPO1130 (SEQ ID NO: 406), GHPO1134 (SEQ ID NO: 408), GHPO1161 (SEQ ID NO: 410), GHPO1166 (SEQ ID NO: 412), GHPO1170 (SEQ ID NO: 414), GHPO1175 (SEQ ID NO: 416), GHPO1181 (SEQ ID NO: 418), GHPO1186 (SEQ ID NO: 420), GHPO1188 (SEQ ID NO: 422), GHPO1191 (SEQ ID NO: 424), GHPO1193 (SEQ ID NO: 426), GHPO1196 (SEQ ID NO: 428), GHPO1204 (SEQ ID NO: 430), GHPO1210 (SEQ ID NO: 432), GHPO1211 (SEQ ID NO: 434), GHPO1216 (SEQ ID NO: 436), GHPO1218 (SEQ ID NO: 438), GHPO1220 (SEQ ID NO: 440), GHPO1223 (SEQ ID NO: 442), GHPO1226 (SEQ ID NO: 444), GHPO1240 (SEQ ID NO: 446), GHPO1246 (SEQ ID NO: 448), GHPO1251 (SEQ ID NO: 450), GHPO1252 (SEQ ID NO: 452), GHPO1261 (SEQ ID NO: 454), GHPO1265 (SEQ ID NO: 456), GHPO1267 (SEQ ID NO: 458), GHPO1278 (SEQ ID NO: 460), GHPO1282 (SEQ ID NO: 462), GHPO1283 (SEQ ID NO: 464), GHPO1287 (SEQ ID NO: 466), GHPO1292 (SEQ ID NO: 468), GHPO1293 (SEQ ID NO: 470), GHPO1302 (SEQ ID NO: 472), GHPO1309 (SEQ ID NO: 474), GHPO1317 (SEQ ID NO: 476), GHPO1318 (SEQ ID NO: 478), GHPO1321 (SEQ ID NO: 480), GHPO1325 (SEQ ID NO: 482), GHPO1341 (SEQ ID NO: 484), GHPO1351 (SEQ ID NO: 486), GHPO1354 (SEQ ID NO: 488), GHPO1363 (SEQ ID NO: 490), GHPO1371 (SEQ ID NO: 492), GHPO1381 (SEQ ID NO: 494), GHPO1401 (SEQ ID NO: 496), GHPO1402 (SEQ ID NO: 498), GHPO1403 (SEQ ID NO: 500), GHPO1408 (SEQ ID NO: 502), GHPO1416 (SEQ ID NO: 504), GHPO1420 (SEQ ID NO: 506), GHPO1428 (SEQ ID NO: 508), GHPO1437 (SEQ ID NO: 510), GHPO1439 (SEQ ID NO: 512), GHPO1460 (SEQ ID NO: 514), GHPO1463 (SEQ ID NO: 516), GHPO1472 (SEQ ID NO: 518), GHPO1474 (SEQ ID NO: 520), GHPO1484 (SEQ ID NO: 522), GHPO1489 (SEQ ID NO: 524), GHPO1494 (SEQ ID NO: 526), GHPO1495 (SEQ ID NO: 528), GHPO1498 (SEQ ID NO: 530), GHPO1499 (SEQ ID NO: 532), GHPO1500 (SEQ ID NO: 534), GHPO1503 (SEQ ID NO: 536), GHPO1504 (SEQ ID NO: 538), GHPO1510 (SEQ ID NO: 540), GHPO1518 (SEQ ID NO: 542), GHPO1533 (SEQ ID NO: 544), GHPO1541 (SEQ ID NO: 546), GHPO1544 (SEQ ID NO: 548), GHPO1548 (SEQ ID NO: 550), GHPO1565 (SEQ ID NO: 552), GHPO1575 (SEQ ID NO: 554), GHPO1582 (SEQ ID NO: 556), GHPO1595 (SEQ ID NO: 558), GHPO1597 (SEQ ID NO: 560), GHPO1599 (SEQ ID NO: 562), GHPO1601 (SEQ ID NO: 564), GHPO1609 (SEQ ID NO: 566), GHPO1613 (SEQ ID NO: 568), GHPO1614 (SEQ ID NO: 570), GHPO1626 (SEQ ID NO: 572), GHPO1628 (SEQ ID NO: 574), GHPO1639 (SEQ ID NO: 576), GHPO1640 (SEQ ID NO: 578), GHPO1641 (SEQ ID NO: 580), GHPO1646 (SEQ ID NO: 582), GHPO1662 (SEQ ID NO: 584), GHPO1667 (SEQ ID NO: 586), GHPO1668 (SEQ ID NO: 588), GHPO1670 (SEQ ID NO: 590), GHPO1671 (SEQ ID NO: 592), GHPO1672 (SEQ ID NO: 594), GHPO1678 (SEQ ID NO: 596), GHPO1684 (SEQ ID NO: 598), GHPO1695 (SEQ ID NO: 600), GHPO1697 (SEQ ID NO: 602), GHPO1701 (SEQ ID NO: 604), GHPO1719 (SEQ ID NO: 606), GHPO1723 (SEQ ID NO: 608), GHPO1732 (SEQ ID NO: 610), GHPO1739 (SEQ ID NO: 612), GHPO1741 (SEQ ID NO: 614), GHPO1747 (SEQ ID NO: 616), GHPO1749 (SEQ ID NO: 618), GHPO1750 (SEQ ID NO: 620), GHPO1751 (SEQ ID NO: 622), GHPO1755 (SEQ ID NO: 624), GHPO1771 (SEQ ID NO: 626), GHPO1786 (SEQ ID NO: 628), and GHPO1789 (SEQ ID NO: 630), which can be used, e.g., in methods to prevent, treat, or diagnose Helicobacter infection. The sequences of polynucleotides that encode these polypeptides are shown in the sequence listing (odd numbers, up to SEQ ID NO: 629). Those skilled in the art will understand that the invention also includes polynucleotide molecules that encode mutants and derivatives of these polypeptides, which can result from the addition, deletion, or substitution of non-essential amino acids, as is described further below.

In addition to the polynucleotide molecules described above, the invention includes the corresponding polypeptides (i.e., polypeptides encoded by the polynucleotide molecules of the invention, or fragments thereof), and monospecific antibodies that specifically bind to these polypeptides. The polypeptides of the invention include those having the amino acid sequences shown in the sequence listing (even numbers, up to SEQ ID NO: 630), as well as mature forms of proteins having sequences shown in the sequence listing in their unprocessed forms, and fragments thereof.

The present invention has many applications and includes expression cassettes, vectors, and cells transformed or transfected with the polynucleotides of the invention. Accordingly, the present invention provides (i) methods for producing polypeptides of the invention in recombinant host systems and related expression cassettes, vectors, and transformed or transfected cells; (ii) live vaccine vectors, such as pox virus, Salmonella typhimurium, and Vibrio cholerae vectors, that contain polynucleotides of the invention (such vaccine vectors being useful in, e.g., methods for preventing or treating Helicobacter infection) in combination with a diluent or carrier, and related pharmaceutical compositions and associated therapeutic and/or prophylactic methods; (iii) therapeutic and/or prophylactic methods involving administration of polynucleotide molecules, either in a naked form or formulated with a delivery vehicle, polypeptides or mixtures of polypeptides, or monospecific antibodies of the invention, and related pharmaceutical compositions; (iv) methods for detecting the presence of Helicobacter in biological samples, which can involve the use of polynucleotide molecules, monospecific antibodies, or polypeptides of the invention; and (v) methods for purifying polypeptides of the invention by antibody-based affinity chromatography.

### DETAILED DESCRIPTION

Open reading frames (ORFs) encoding new polypeptides, designated GHPO7 (SEQ ID NO: 2), GHPO8 (SEQ ID NO: 4), GHPO9 (SEQ ID NO: 6), GHPO10 (SEQ ID NO: 8), GHPO12 (SEQ ID NO: 10), GHPO25 (SEQ ID NO: 12), GHPO27 (SEQ ID NO: 14), GHPO29 (SEQ ID NO: 16), GHPO30 (SEQ ID NO: 18), GHPO37 (SEQ ID NO: 20), GHPO49 (SEQ ID NO: 22), GHPO51 (SEQ ID NO: 24), GHPO54 (SEQ ID NO: 26), GHPO65 (SEQ ED NO: 28), GHPO66 (SEQ ID NO: 30), GHPO68 (SEQ ID NO: 32), GHPO07 (SEQ ID NO: 34), GHPO77 (SEQ ID NO: 36), GHPO83 (SEQ ID NO: 38), GHPO85 (SEQ ID NO: 40), GHPO87 (SEQ ID NO: 42), GHPO09 (SEQ ID NO: 44), GHPO92 (SEQ ID NO: 46), GHPO96 (SEQ ID NO: 48), GHPO97 (SEQ ID NO: 50), GHPO111 (SEQ ID NO: 52), GHPO115 (SEQ ID NO: 54), GHPO117 (SEQ ID NO: 56), GHPO123 (SEQ ID NO: 58), GHPO124 (SEQ ID NO: 60), GHPO126 (SEQ ID NO: 62), GHPO127 (SEQ ID NO: 64), GHPO128 (SEQ ID NO: 66), GHPO131 (SEQ ID NO: 68), GHPO033 (SEQ ID NO: 70), GHPO014 (SEQ ID NO: 72), GHPO014 (SEQ ID NO: 74), GHPO045 (SEQ ID NO: 76), GHPO047 (SEQ ID NO: 78), GHPO066 (SEQ ID NO: 80), GHPO181 (SEQ ID NO: 82), GHPO187 (SEQ ID NO: 84), GHPO188 (SEQ ID NO: 86), GHPO192 (SEQ ID NO: 88), GHPO202 (SEQ ID NO: 90), GHPO204 (SEQ ID NO: 92), GHPO205 (SEQ ID NO: 94), GHPO212 (SEQ ID NO: 96), GHPO218 (SEQ ID NO: 98), GHPO226 (SEQ ID NO: 100), GHPO231 (SEQ ID NO: 102), GHPO236 (SEQ ID NO: 104), GHPO239 (SEQ ID NO: 106), GHPO245 (SEQ ID NO: 108), GHPO246 (SEQ ID NO: 110), GHPO248 (SEQ ID NO: 112), GHPO253 (SEQ ID NO: 114), GHPO265 (SEQ ID NO: 116), GHPO266 (SEQ ID NO: 118), GHPO271 (SEQ ID NO: 120), GHPO272 (SEQ ID NO: 122), GHPO286 (SEQ ID NO: 124), GHPO291 (SEQ ID NO: 126), GHPO292 (SEQ ID NO: 128), GHPO297 (SEQ ID NO: 130), GHPO304 (SEQ ID NO: 132), GHPO307 (SEQ ID NO: 134), GHPO324 (SEQ ID NO: 136), GHPO326 (SEQ ID NO: 138), GHPO331 (SEQ ID NO: 140), GHPO343 (SEQ ID NO: 142), GHPO345 (SEQ ID NO: 144), GHPO346 (SEQ ID NO: 146), GHPO352 (SEQ ID NO: 148), GHPO355 (SEQ ID NO: 150), GHPO363 (SEQ ID NO: 152), GHPO369 (SEQ ID NO: 154), GHPO376 (SEQ ID NO: 156), GHPO378 (SEQ ID NO: 158), GHPO388 (SEQ ID NO: 160), GHPO396 (SEQ ID NO: 162), GHPO403 (SEQ ID NO: 164), GHPO410 (SEQ ID NO: 166), GHPO415 (SEQ ID NO: 168), GHPO421 (SEQ ID NO: 170), GHPO439 (SEQ ID NO: 172), GHPO441 (SEQ ID NO: 174), GHPO443 (SEQ ID NO: 176), GHPO453 (SEQ ID NO: 178), GHPO455 (SEQ ID NO: 180), GHPO464 (SEQ ID NO: 182), GHPO467 (SEQ ID NO: 184), GHPO468 (SEQ ID NO: 186), GHPO470 (SEQ ID NO: 188), GHPO486 (SEQ ID NO: 190), GHPO487 (SEQ ID NO: 192), GHPO488 (SEQ ID NO: 194), GHPO489 (SEQ ID NO: 196), GHPO498 (SEQ ID NO: 198), GHPO501 (SEQ ID NO: 200), GHPO504 (SEQ ID NO: 202), GHPO512 (SEQ ID NO: 204), GHPO517 (SEQ ID NO: 206), GHPO520 (SEQ ID NO: 208), GHPO528 (SEQ ID NO: 210), GHPO530 (SEQ ID NO: 212), GHPO532 (SEQ ID NO: 214), GHPO548 (SEQ ID NO: 216), GHPO561 (SEQ ID NO: 218), GHPO564 (SEQ ID NO: 220), GHPO572 (SEQ ID NO: 222), GHPO573 (SEQ ID NO: 224), GHPO574 (SEQ ID NO: 226), GHPO577 (SEQ ID NO: 228), GHPO579 (SEQ ID NO: 230), GHPO583 (SEQ ID NO: 232), GHPO588 (SEQ ID NO: 234), GHPO593 (SEQ ID NO: 236), GHPO597 (SEQ ID NO: 238), GHPO598 (SEQ ID NO: 240), GHPO604 (SEQ ID NO: 242), GHPO606 (SEQ ID NO: 244), GHPO611 (SEQ ID NO: 246), GHPO612 (SEQ ID NO: 248), GHPO615 (SEQ ID NO: 250), GHPO632 (SEQ ID NO: 252), GHPO633 (SEQ ID NO: 254), GHPO637 (SEQ ID NO: 256), GHPO651 (SEQ ID NO: 258), GHPO663 (SEQ ID NO: 260), GHPO686 (SEQ ID NO: 262), GHPO693 (SEQ ID NO: 264), GHPO698 (SEQ ID NO: 266), GHPO703 (SEQ ID NO: 268), GHPO704 (SEQ ID NO: 270), GHPO705 (SEQ ID NO: 272), GHPO707 (SEQ ID NO: 274), GHPO721 (SEQ ID NO: 276), GHPO727 (SEQ ID NO: 278), GHPO728 (SEQ ID NO: 280), GHPO733 (SEQ ID NO: 282), GHPO758 (SEQ ID NO: 284), GHPO763 (SEQ ID NO: 286), GHPO771 (SEQ ID NO: 288), GHPO774 (SEQ ID NO: 290), GHPO776 (SEQ ID NO: 292), GHPO783 (SEQ ID NO: 294), GHPO800 (SEQ ID NO: 296), GHPO806 (SEQ ID NO: 298), GHPO807 (SEQ ID NO: 300), GHPO808 (SEQ ID NO: 302), GHPO809 (SEQ ID NO: 304), GHPO811 (SEQ ID NO: 306), GHPO815 (SEQ ID NO: 308), GHPO819 (SEQ ID NO: 310), GHPO841 (SEQ ID NO: 312), GHPO843 (SEQ ID NO: 314), GHPO846 (SEQ ID NO: 316), GHPO875 (SEQ ID NO: 318), GHPO892 (SEQ ID NO: 320), GHPO902 (SEQ ID NO: 322), GHPO904 (SEQ ID NO: 324), GHPO906 (SEQ ID NO: 326), GHPO908 (SEQ ID NO: 328), GHPO921 (SEQ ID NO: 330), GHPO923 (SEQ ID NO: 332), GHPO926 (SEQ ID NO: 334), GHPO933 (SEQ ID NO: 336), GHPO939 (SEQ ID NO: 338), GHPO940 (SEQ ID NO: 340), GHPO943 (SEQ ID NO: 342), GHPO951 (SEQ ID NO: 344), GHPO961 (SEQ ID NO: 346), GHPO965 (SEQ ID NO: 348), GHPO990 (SEQ ID NO: 350), GHPO991 (SEQ ID NO: 352), GHPO998 (SEQ ID NO: 354), GHPO1001 (SEQ ID NO: 356), GHPO1005 (SEQ ID NO: 358), GHPO1033 (SEQ ID NO: 360), GHPO1039 (SEQ ID NO: 362), GHPO1041 (SEQ ID NO: 364), GHPO1043 (SEQ ID NO: 366), GHPO1044 (SEQ ID NO: 368), GHPO1051 (SEQ ID NO: 370), GHPO1058 (SEQ ID NO: 372), GHPO1060 (SEQ ID NO: 374), GHPO1075 (SEQ ID NO: 376), GHPO1077 (SEQ ID NO: 378), GHPO1082 (SEQ ID NO: 380), GHPO1083 (SEQ ID NO: 382), GHPO1086 (SEQ ID NO: 384), GHPO1087 (SEQ ID NO: 386), GHPO1090 (SEQ ID NO: 388), GHPO1097 (SEQ ID NO: 390), GHPO1098 (SEQ ID NO: 392), GHPO1103 (SEQ ID NO: 394), GHPO1113 (SEQ ID NO: 396), GHPO1116 (SEQ ID NO: 398), GHPO1123 (SEQ ID NO: 400), GHPO1125 (SEQ ID NO: 402), GHPO1129 (SEQ ID NO: 404), GHPO1130 (SEQ ID NO: 406), GHPO1134 (SEQ ID NO: 408), GHPO1161 (SEQ ID NO: 410), GHPO1166 (SEQ ID NO: 412), GHPO1170 (SEQ ID NO: 414), GHPO1175 (SEQ ID NO: 416), GHPO1181 (SEQ ID NO: 418), GHPO1186 (SEQ ID NO: 420), GHPO1188 (SEQ ID NO: 422), GHPO1191 (SEQ ID NO: 424), GHPO1193 (SEQ ID NO: 426), GHPO1196 (SEQ ID NO: 428), GHPO1204 (SEQ ID NO: 430), GHPO1210 (SEQ ID NO: 432), GHPO1211 (SEQ ID NO: 434), GHPO1216 (SEQ ID NO: 436), GHPO1218 (SEQ ID NO: 438), GHPO1220 (SEQ ID NO: 440), GHPO1223 (SEQ ID NO: 442), GHPO1226 (SEQ ID NO: 444), GHPO1240 (SEQ ID NO: 446), GHPO1246 (SEQ ID NO: 448), GHPO1251 (SEQ ID NO: 450), GHPO1252 (SEQ ID NO: 452), GHPO1261 (SEQ ID NO: 454), GHPO1265 (SEQ ID NO: 456), GHPO1267 (SEQ ID NO: 458), GHPO1278 (SEQ ID NO: 460), GHPO1282 (SEQ ID NO: 462), GHPO1283 (SEQ ID NO: 464), GHPO1287 (SEQ ID NO: 466), GHPO1292 (SEQ ID NO: 468), GHPO1293 (SEQ ID NO: 470), GHPO1302 (SEQ ID NO: 472), GHPO1309 (SEQ ID NO: 474), GHPO1317 (SEQ ID NO: 476), GHPO1318 (SEQ ID NO: 478), GHPO1321 (SEQ ID NO: 480), GHPO1325 (SEQ ID NO: 482), GHPO1341 (SEQ ID NO: 484), GHPO1351 (SEQ ID NO: 486), GHPO1354 (SEQ ID NO: 488), GHPO1363 (SEQ ID NO: 490), GHPO1371 (SEQ ID NO: 492), GHPO1381 (SEQ ID NO: 494), GHPO1401 (SEQ ID NO: 496), GHPO1402 (SEQ ID NO: 498), GHPO1403 (SEQ ID NO: 500), GHPO1408 (SEQ ID NO: 502), GHPO1416 (SEQ ID NO: 504), GHPO1420 (SEQ ID NO: 506), GHPO1428 (SEQ ID NO: 508), GHPO1437 (SEQ ID NO: 510), GHPO1439 (SEQ ID NO: 512), GHPO1460 (SEQ ID NO: 514), GHPO1463 (SEQ ID NO: 516), GHPO1472 (SEQ ID NO: 518), GHPO1474 (SEQ ID NO: 520), GHPO1484 (SEQ ID NO: 522), GHPO1489 (SEQ ID NO: 524), GHPO1494 (SEQ ID NO: 526), GHPO1495 (SEQ ID NO: 528), GHPO1498 (SEQ ID NO: 530), GHPO1499 (SEQ ID NO: 532), GHPO1500 (SEQ ID NO: 534), GHPO1503 (SEQ ID NO: 536), GHPO1504 (SEQ ID NO: 538), GHPO1510 (SEQ ID NO: 540), GHPO1518 (SEQ ID NO: 542), GHPO1533 (SEQ ID NO: 544), GHPO1541 (SEQ ID NO: 546), GHPO1544 (SEQ ID NO: 548), GHPO1548 (SEQ ID NO: 550), GHPO1565 (SEQ ID NO: 552), GHPO1575 (SEQ ID NO: 554), GHPO1582 (SEQ ID NO: 556), GHPO1595 (SEQ ID NO: 558), GHPO1597 (SEQ ID NO: 560), GHPO1599 (SEQ ID NO: 562), GHPO1601 (SEQ ID NO: 564), GHPO1609 (SEQ ID NO: 566), GHPO1613 (SEQ ID NO: 568), GHPO1614 (SEQ ID NO: 570), GHPO1626 (SEQ ID NO: 572), GHPO1628 (SEQ ID NO: 574), GHPO1639 (SEQ ID NO: 576), GHPO1640 (SEQ ID NO: 578), GHPO1641 (SEQ ID NO: 580), GHPO1646 (SEQ ID NO: 582), GHPO1662 (SEQ ID NO: 584), GHPO1667 (SEQ ID NO: 586), GHPO1668 (SEQ ID NO: 588), GHPO1670 (SEQ ID NO: 590), GHPO1671 (SEQ ID NO: 592), GHPO1672 (SEQ ID NO: 594), GHPO1678 (SEQ ID NO: 596), GHPO1684 (SEQ ID NO: 598), GHPO1695 (SEQ ID NO: 600), GHPO1697 (SEQ ID NO: 602), GHPO1701 (SEQ ID NO: 604), GHPO1719 (SEQ ID NO: 606), GHPO1723 (SEQ ID NO: 608), GHPO1732 (SEQ ID NO: 610), GHPO1739 (SEQ ID NO: 612), GHPO1741 (SEQ ID NO: 614), GHPO1747 (SEQ ID NO: 616), GHPO1749 (SEQ ID NO: 618), GHPO1750 (SEQ ID NO: 620), GHPO1751 (SEQ ID NO: 622), GHPO1755 (SEQ ID NO: 624), GHPO1771 (SEQ ID NO: 626), GHPO1786 (SEQ ID NO: 628), and GHPO1789 (SEQ ID NO: 630), have been identified in the H. pylori genome. These polypeptides can be used, for example, in vaccination methods for preventing or treating Helicobacter infection. Some of the new polypeptides are secreted polypeptides that can be produced in their mature forms (i.e., as polypeptides that have been exported through class II or class III secretion pathways) or as precursors that include signal peptides, which can be removed in the course of excretion/secretion by cleavage at the N-terminal end of the mature form. (The cleavage site is located at the C-terminal end of the signal peptide, adjacent to the mature form.)

According to a first aspect of the invention, there are provided isolated polynucleotides that encode the precursor and mature forms of the Helicobacter GHPO proteins listed above. Examples of such polynucleotides are those encoding GHPO7 (SEQ ID NO: 1), GHPO8 (SEQ ID NO: 3), GHPO9 (SEQ ID NO: 5), GHPO10 (SEQ ID NO: 7), GHPO12 (SEQ ID NO: 9), GHPO25 (SEQ ID NO: 11), GHPO27 (SEQ ID NO: 13), GHPO29 (SEQ ID NO: 15), GHPO30 (SEQ ID NO: 17), GHPO37 (SEQ ID NO: 19), GHPO49 (SEQ ID NO: 21), GHPO51 (SEQ ID NO: 23), GHPO54 (SEQ ID NO: 25), GHPO65 (SEQ ID NO: 27), GHPO66 (SEQ ID NO: 29), GHPO68 (SEQ ID NO: 31), GHPO70 (SEQ ID NO: 33), GHPO77 (SEQ ID NO: 35), GHPO83 (SEQ ID NO: 37), GHPO85 (SEQ ID NO: 39), GHPO87 (SEQ ID NO: 41), GHPO91 (SEQ ID NO: 43), GHPO92 (SEQ ID NO: 45), GHPO96 (SEQ ID NO: 47), GHPO97 (SEQ ID NO: 49), GHPO111 (SEQ ID NO: 51), GHPO115 (SEQ ID NO: 53), GHPO117 (SEQ ID NO: 55), GHPO123 (SEQ ID NO: 57), GHPO124 (SEQ ID NO: 59), GHPO126 (SEQ ID NO: 61), GHPO127 (SEQ ID NO: 63), GHPO128 (SEQ ID NO: 65), GHPO131 (SEQ ID NO: 67), GHPO133 (SEQ ID NO: 69), GHPO140 (SEQ ID NO: 71), GHPO141 (SEQ ID NO: 73), GHPO145 (SEQ ID NO: 75), GHPO147 (SEQ ID NO: 77), GHPO166 (SEQ ID NO: 79), GHPO181 (SEQ ID NO: 81), GHPO187 (SEQ ID NO: 83), GHPO188 (SEQ ID NO: 85), GHPO192 (SEQ ID NO: 87), GHPO202 (SEQ ID NO: 89), GHPO204 (SEQ ID NO: 91), GHPO205 (SEQ ID NO: 93), GHPO212 (SEQ ID NO: 95), GHPO218 (SEQ ID NO: 97), GHPO226 (SEQ ID NO: 99), GHPO231 (SEQ ID NO:l01), GHPO236 (SEQ ID NO: 103), GHPO239 (SEQ ID NO: 105), GHPO245 (SEQ ID NO: 107), GHPO246 (SEQ ID NO: 109), GHPO248 (SEQ ID NO: 111), GHPO253 (SEQ ID NO: 113), GHPO265 (SEQ ID NO: 115), GHPO266 (SEQ ID NO: 117), GHPO271 (SEQ ID NO: 119), GHPO272 (SEQ ID NO: 121), GHPO286 (SEQ ID NO: 123), GHPO291 (SEQ ID NO: 125), GHPO292 (SEQ ID NO: 127), GHPO297 (SEQ ID NO: 129), GHPO304 (SEQ ID NO: 131), GHPO307 (SEQ ID NO: 133), GHPO324 (SEQ ID NO: 135), GHPO326 (SEQ ID NO: 137), GHPO331 (SEQ ID NO: 139), GHPO343 (SEQ ID NO: 141), GHPO345 (SEQ ID NO: 143), GHPO346 (SEQ ID NO: 145), GHPO0352 (SEQ ID NO: 147), GHPO355 (SEQ ID NO: 149), GHPO363 (SEQ ID NO: 151), GHPO369 (SEQ ID NO: 153), GHPO376 (SEQ ID NO: 155), GHPO378 (SEQ ID NO: 157), GHPO388 (SEQ ID NO: 159), GHPO396 (SEQ ID NO: 161), GHPO403 (SEQ ID NO: 163), GHPO410 (SEQ ID NO: 165), GHPO415 (SEQ ID NO: 167), GHPO421 (SEQ ID NO: 169), GHPO439 (SEQ ID NO: 171), GHPO441 (SEQ ID NO: 173), GHPO443 (SEQ ID NO: 175), GHPO453 (SEQ ID NO: 177), GHPO455 (SEQ ID NO: 179), GHPO464 (SEQ ID NO: 181), GHPO467 (SEQ ID NO: 183), GHPO468 (SEQ ID NO: 185), GHPO470 (SEQ ID NO: 187), GHPO486 (SEQ ID NO: 189), GHPO487 (SEQ ID NO: 191), GHPO488 (SEQ ID NO: 193), GHPO489 (SEQ ID NO: 195), GHPO498 (SEQ ID NO: 197), GHPO501 (SEQ ID NO: 199), GHPO504 (SEQ ID NO: 201), GHPO512 (SEQ ID NO: 203), GHPO517 (SEQ ID NO: 205), GHPO520 (SEQ ID NO: 207), GHPO528 (SEQ ID NO: 209), GHPO530 (SEQ ID NO: 211), GHPO532 (SEQ ID NO: 213), GHPO548 (SEQ ID NO: 215), GHPO561 (SEQ ID NO: 217), GHPO564 (SEQ ID NO: 219), GHPO572 (SEQ ID NO: 221), GHPO573 (SEQ ID NO: 223), GHPO574 (SEQ ID NO: 225), GHPO577 (SEQ ID NO: 227), GHPO579 (SEQ ID NO: 229), GHPO583 (SEQ ID NO: 231), GHPO588 (SEQ ID NO: 233), GHPO593 (SEQ ID NO: 235), GHPO597 (SEQ ID NO: 237), GHPO598 (SEQ ID NO: 239), GHPO604 (SEQ ID NO: 241), GHPO606 (SEQ ID NO: 243), GHPO611 (SEQ ID NO: 245), GHPO612 (SEQ ID NO: 247), GHPO615 (SEQ ID NO: 249), GHPO632 (SEQ ID NO: 251), GHPO633 (SEQ ID NO: 253), GHPO637 (SEQ ID NO: 255), GHPO651 (SEQ ID NO: 257), GHPO663 (SEQ ID NO: 259), GHPO686 (SEQ ID NO: 261), GHPO693 (SEQ ID NO: 263), GHPO698 (SEQ ID NO: 265), GHPO703 (SEQ ID NO: 267), GHPO704 (SEQ ID NO: 269), GHPO705 (SEQ ID NO: 271), GHPO707 (SEQ ID NO: 273), GHPO721 (SEQ ID NO: 275), GHPO727 (SEQ ID NO: 277), GHPO728 (SEQ ID NO: 279), GHPO733 (SEQ ID NO: 281), GHPO758 (SEQ ID NO: 283), GHPO763 (SEQ ID NO: 285), GHPO771 (SEQ ID NO: 287), GHPO774 (SEQ ID NO: 289), GHPO776 (SEQ ID NO: 291), GHPO783 (SEQ ID NO: 293), GHPO800 (SEQ ID NO: 295), GHPO806 (SEQ ID NO: 297), GHPO807 (SEQ ID NO: 299), GHPO808 (SEQ ID NO: 301), GHPO809 (SEQ ID NO: 303), GHPO811 (SEQ ID NO: 305), GHPO815 (SEQ ID NO: 307), GHPO819 (SEQ ID NO: 309), GHPO841 (SEQ ID NO: 311), GHPO843 (SEQ ID NO: 313), GHPO846 (SEQ ID NO: 315), GHPO875 (SEQ ID NO: 317), GHPO892 (SEQ ID NO: 319), GHPO902 (SEQ ID NO: 321), GHPO904 (SEQ ID NO: 323), GHPO906 (SEQ ID NO: 325), GHPO908 (SEQ ID NO: 327), GHPO921 (SEQ ID NO: 329), GHPO923 (SEQ ID NO: 331), GHPO926 (SEQ ID NO: 333), GHPO933 (SEQ ID NO: 335), GHPO939 (SEQ ID NO: 337), GHPO940 (SEQ ID NO: 339), GHPO943 (SEQ ID NO: 341), GHPO951 (SEQ ID NO: 343), GHPO961 (SEQ ID NO: 345), GHPO965 (SEQ ID NO: 347), GHPO990 (SEQ ID NO: 349), GHPO991 (SEQ ID NO: 351), GHPO998 (SEQ ID NO: 353), GHPO1001 (SEQ ID NO: 355), GHPO1005 (SEQ ID NO: 357), GHPO1033 (SEQ ID NO: 359), GHPO1039 (SEQ ID NO: 361), GHPO1041 (SEQ ID NO: 363), GHPO1043 (SEQ ID NO: 365), GHPO1044 (SEQ ID NO: 367), GHPO1051 (SEQ ID NO: 369), GHPO1058 (SEQ ID NO: 371), GHPO1060 (SEQ ID NO: 373), GHPO1075 (SEQ ID NO: 375), GHPO1077 (SEQ ID NO: 377), GHPO1082 (SEQ ID NO: 379), GHPO1083 (SEQ ID NO: 381), GHPO1086 (SEQ ID NO: 383), GHPO1087 (SEQ ID NO: 385), GHPO1090 (SEQ ID NO: 387), GHPO1097 (SEQ ID NO: 389), GHPO1098 (SEQ ID NO: 391), GHPO1103 (SEQ ID NO: 393), GHPO1113 (SEQ ID NO: 395), GHPO1116 (SEQ ID NO: 397), GHPO1123 (SEQ ID NO: 399), GHPO1125 (SEQ ID NO: 401), GHPO1129 (SEQ ID NO: 403), GHPO1130 (SEQ ID NO: 405), GHPO1134 (SEQ ID NO: 407), GHPO1161 (SEQ ID NO: 409), GHPO1166 (SEQ ID NO: 411), GHPO1170 (SEQ ID NO: 413), GHPO1175 (SEQ ID NO: 415), GHPO1181 (SEQ ID NO: 417), GHPO1186 (SEQ ID NO: 419), GHPO1188 (SEQ ID NO: 421), GHPO1191 (SEQ ID NO: 423), GHPO1193 (SEQ ID NO: 425), GHPO1196 (SEQ ID NO: 427), GHPO1204 (SEQ ID NO: 429), GHPO1210 (SEQ ID NO: 431), GHPO1211 (SEQ ID NO: 433), GHPO1216 (SEQ ID NO: 435), GHPO1218 (SEQ ID NO: 437), GHPO1220 (SEQ ID NO: 439), GHPO1223 (SEQ ID NO: 441), GHPO1226 (SEQ ID NO: 443), GHPO1240 (SEQ ID NO: 445), GHPO1246 (SEQ ID NO: 447), GHPO)1251 (SEQ ID NO: 449), GHPO1252 (SEQ ID NO: 451), GHPO1261 (SEQ ID NO: 453), GHPO1265 (SEQ ID NO: 455), GHPO1267 (SEQ ID NO: 457), GHPO1278 (SEQ ID NO: 459), GHPO1282 (SEQ ID NO: 461), GHPO1283 (SEQ ID NO: 463), GHPO1287 (SEQ ID NO: 465), GHPO1292 (SEQ ID NO: 467), GHPO1293 (SEQ ID NO: 469), GHPO1302 (SEQ ID NO: 471), GHPO1309 (SEQ ID NO: 473), GHPO1317 (SEQ ID NO: 475), GHPO1318 (SEQ ID NO: 477), GHPO1321 (SEQ ID NO: 479), GHPO1325 (SEQ ID NO: 481), GHPO1341 (SEQ ID NO: 483), GHPO1351 (SEQ ID NO: 485), GHPO1354 (SEQ ID NO: 487), GHPO1363 (SEQ ID NO: 489), GHPO1371 (SEQ ID NO: 491), GHPO1381 (SEQ ID NO: 493), GHPO1401 (SEQ ID NO: 495), GHPO1402 (SEQ ID NO: 497), GHPO1403 (SEQ ID NO: 499), GHPO1408 (SEQ ID NO: 501), GHPO1416 (SEQ ID NO: 503), GHPO1420 (SEQ ID NO: 505), GHPO1428 (SEQ ID NO: 507), GHPO1437 (SEQ ID NO: 509), GHPO1439 (SEQ ID NO: 511), GHPO1460 (SEQ ID NO: 513), GHPO1463 (SEQ ID NO: 515), GHPO1472 (SEQ ID NO: 517), GHPO1474 (SEQ ID NO: 519), GHPO1484 (SEQ ID NO: 521), GHPO1489 (SEQ ID NO: 523), GHPO1494 (SEQ ID NO: 525), GHPO1495 (SEQ ID NO: 527), GHPO1498 (SEQ ID NO: 529), GHPO1499 (SEQ ID NO: 531), GHPO1500 (SEQ ID NO: 533), GHPO1503 (SEQ ID NO: 535), GHPO1504 (SEQ ID NO: 537), GHPO1510 (SEQ ID NO: 539), GHPO1518 (SEQ ID NO: 541), GHPO1533 (SEQ ID NO: 543), GHPO1541 (SEQ ID NO: 545), GHPO1544 (SEQ ID NO: 547), GHPO1548 (SEQ ID NO: 549), GHPO1565 (SEQ ID NO: 551), GHPO1575 (SEQ ID NO: 553), GHPO1582 (SEQ ID NO: 555), GHPO1595 (SEQ ID NO: 557), GHPO1597 (SEQ ID NO: 559), GHPO1599 (SEQ ID NO: 561), GHPO1601 (SEQ ID NO: 563), GHPO1609 (SEQ ID NO: 565), GHPO1613 (SEQ ID NO: 567), GHPO1614 (SEQ ID NO: 569), GHPO1626 (SEQ ID NO: 571), GHPO1628 (SEQ ID NO: 573), GHPO1639 (SEQ ID NO: 575), GHPO1640 (SEQ ID NO: 577), GHPO1641 (SEQ ID NO: 579), GHPO1646 (SEQ ID NO: 581), GHPO1662 (SEQ ID NO: 583), GHPO1667 (SEQ ID NO: 585), GHPO1668 (SEQ ID NO: 587), GHPO1670 (SEQ ID NO: 589), GHPO1671 (SEQ ID NO: 591), GHPO1672 (SEQ ID NO: 593), GHPO1678 (SEQ ID NO: 595), GHPO1684 (SEQ ID NO: 597), GHPO1695 (SEQ ID NO: 599), GHPO1697 (SEQ ID NO: 601), GHPO1701 (SEQ ID NO: 603), GHPO1719 (SEQ ID NO: 605), GHPO1723 (SEQ ID NO: 607), GHPO732 (SEQ ID NO: 609), GHPO1739 (SEQ ID NO: 611), GHPO1741 (SEQ ID NO: 613), GHPO1747 (SEQ ID NO: 615), GHPO1749 (SEQ ID NO: 617), GHPO1750 (SEQ ID NO: 619), GHPO1751 (SEQ ID NO: 621), GHPO1755 (SEQ ID NO: 623), GHPO1771 (SEQ ID NO: 625), GHPO1786 (SEQ ID NO: 627), and GHPO1789 (SEQ ID NO: 629).

An isolated polynucleotide of the invention encodes (i) a polypeptide having an amino acid sequence that is homologous to a Helicobacter amino acid sequence of a polypeptide, the Helicobacter amino acid sequence being selected from the group consisting of the amino acid sequences shown in the sequence listing (even numbers, up to SEQ ID NO: 630), or (ii) a derivative of the polypeptide.

In addition to the fuill-length polypeptides encoded by the polynucleotides of the invention, as set forth above, polynucleotides included in the invention can also encode polypeptides that lack signal sequences, as well as other polypeptide or peptide fragments of the full-length polypeptides.

The term “isolated polynucleotide” is defined as a polynucleotide that is removed from the environment in which it naturally occurs. For example, a naturally-occurring DNA molecule present in the genome of a living bacteria or as part of a gene bank is not isolated, but the same molecule, separated from the remaining part of the bacterial genome, as a result of, e.g., a cloning event (amplification), is “isolated.” Typically, an isolated DNA molecule is free from DNA regions (e.g., coding regions) with which it is immediately contiguous, at the 5′ or 3′ ends, in the naturally occurring genome. Such isolated polynucleotides can be part of a vector or a composition and still be isolated, as such a vector or composition is not part of its natural environment.

A polynucleotide of the invention can consist of RNA or DNA (e.g., cDNA, genomic DNA, or synthetic DNA), or modifications or combinations of RNA or DNA. The polynucleotide can be double-stranded or single-stranded and, if single-stranded, can be the coding (sense) strand or the non-coding (anti-sense) strand. The sequences that encode polypeptides of the invention, as shown in the sequence listing (even numbers, up to SEQ ID NO: 630), can be (a) the coding sequence as shown in any of the nucleotide sequences of the sequence listing (odd numbers, up to SEQ ID NO: 629); (b) a ribonucleotide sequence derived by transcription of (a); or (c) a different coding sequence that, as a result of the redundancy or degeneracy of the genetic code, encodes the same polypeptides as the polynucleotide molecules having the sequences illustrated in any of the nucleotide sequences of the sequence listing (odd numbers, up to SEQ ID NO: 629). The polypeptide can be one that is naturally secreted or excreted by, e.g., H. felis, H. mustelae, H. heilmanii, or H. pylori.

By “polypeptide” or “protein” is meant any chain of amino acids, regardless of length or post-translational modification (e.g., glycosylation or phosphorylation). Both terms are used interchangeably in the present application.

By “homologous amino acid sequence” is meant an amino acid sequence that differs from an amino acid sequence shown in the sequence listing (even numbers, up to SEQ ID NO: 630), or an amino acid sequence encoded by a nucleotide sequence shown in the sequence listing (odd numbers, up to SEQ ID NO: 629), by one or more non-conservative amino acid substitutions, deletions, or additions located at positions at which they do not destroy the specific antigenicity of the polypeptide. Preferably, such a sequence is at least 75%, more preferably at least 80%, and most preferably at least 90% identical to an amino acid sequence shown in the sequence listing (even numbers, up to SEQ ID NO: 630). Homologous amino acid sequences include sequences that are identical or substantially identical to an amino acid sequence as shown in the sequence listing (even numbers, up to SEQ ID NO: 630). By “amino acid sequence that is substantially identical” is meant a sequence that is at least 90%, preferably at least 95%, more preferably at least 97%, and most preferably at least 99% identical to an amino acid sequence of reference and that differs from the sequence of reference, if at all, by a majority of conservative amino acid substitutions.

Conservative amino acid substitutions typically include substitutions among amino acids of the same class. These classes include, for example, amino acids having uncharged polar side chains, such as asparagine, glutamine, serine, threonine, and tyrosine; amino acids having basic side chains, such as lysine, arginine, and histidine; amino acids having acidic side chains, such as aspartic acid and glutamic acid; and amino acids having nonpolar side chains, such as glycine, alanine, valine, leucine, isoleucine, proline, phenylalanine, methionine, tryptophan, and cysteine.

Homology can be measured using sequence analysis software (e.g., Sequence Analysis Software Package of the Genetics Computer Group, University of Wisconsin Biotechnology Center, 1710 University Avenue, Madison, Wis. 53705). Similar amino acid sequences are aligned to obtain the maximum degree of homology (i.e., identity). To this end, it may be necessary to artificially introduce gaps into the sequence. Once the optimal alignment has been set up, the degree of homology (i.e., identity) is established by recording all of the positions in which the amino acids of both sequences are identical, relative to the total number of positions.

Homologous polynucleotide sequences are defined in a similar way. Preferably, a homologous sequence is one that is at least 45%, more preferably at least 60%, and most preferably at least 85% identical to a coding sequence of any of the nucleotide sequences set forth in the sequence listing (odd numbers, up to SEQ ID NO: 629).

Polypeptides having a sequence homologous to any one of the sequences shown in the sequence listing (even numbers, up to SEQ ID NO: 630), include naturally-occurring allelic variants, as well as mutants or any other non-naturally occurring variants that are analogous in terms of antigenicity, to a polypeptide having a sequence as shown in the sequence listing (even numbers, up to SEQ ID NO: 630).

As is known in the art, an allelic variant is an alternate form of a polypeptide that is characterized as having a substitution, deletion, or addition of one or more amino acids that does not alter the biological function of the polypeptide. By “biological function” is meant a function of the polypeptide in the cells in which it naturally occurs, even if the function is not necessary for the growth or survival of the cells. For example, the biological function of a porin is to allow the entry into cells of compounds present in the extracellular medium. The biological function is distinct from the antigenic function. A polypeptide can have more than one biological function.

Allelic variants are very common in nature. For example, a bacterial species, e.g., H. pylori, is usually represented by a variety of strains that differ from each other by minor allelic variations. Indeed, a polypeptide that fulfills the same biological function in different strains can have an amino acid sequence that is not identical in each of the strains. Such an allelic variation can be equally reflected at the polynucleotide level.

Support for the use of allelic variants of polypeptide antigens comes from, e.g., studies of the Helicobacter urease antigen. The amino acid sequence of Helicobacter urease varies widely from species to species, yet cross-species protection occurs, indicating that the urease molecule, when used as an immunogen, is highly tolerant of amino acid variations. Even among different strains of the single species H. pylori, there are amino acid sequence variations.

For example, although the amino acid sequences of the UreA and UreB subunits of H. pylori and H. felis ureases differ from one another by 26.5% and 11.8%, respectively (Ferrero et al., Molecular Microbiology 9(2):323-333, 1993), it has been shown that H. pylori urease protects mice from H. felis infection (Michetti et al., Gastroenterology 107:1002, 1994). In addition, it has been shown that the individual structural subunits of urease, UreA and UreB, which contain distinct amino acid sequences, are both protective antigens against Helicobacter infection (Michetti et al., supra). Similarly, Cuenca et al. (Gastroenterology 110:1770, 1996) showed that therapeutic immunization of H. mustelae-infected ferrets with H. pylori urease was effective at eradicating H. mustelae infection. Further, several urease variants have been reported to be effective vaccine antigens, including, e.g., recombinant UreA+UreB apoenzyme expressed from pORV142 (UreA and UreB sequences derived from H. pylori strain CPM630; Lee et al., J. Infect. Dis.172:161, 1995); recombinant UreA+UreB apoenzyme expressed from pORV214 (UreA and UreB sequences differ from H. pylori strain CPM630 by one and two amino acid changes, respectively; Lee et al., supra, 1995); a UreA-glutathione-S-transferase fusion protein (UreA sequence from H. pylori strain ATCC 43504; Thomas et al., Acta Gastro-Enterologica Belgica 56:54, 1993); UreA+UreB holoenzyme purified from H. pylori strain NCTC11637 (Marchetti et al., Science 267:1655, 1995); a UreA-MBP fusion protein (UreA from H. pylori strain 85P; Ferrero et al., Infection and Immunity 62:4981, 1994); a UreB-MBP fusion protein (UreB from H. pylori strain 85P; Ferrero et al., supra); a UreA-MBP fusion protein (UreA from H. felis strain ATCC 49179; Ferrero et al., supra); a UreB-MBP fusion protein (UreB from H. felis strain ATCC 49179; Ferrero et al., supra); and a 37 kDa fragment of UreB containing amino acids 220-569 (Dore-Davin et al., “A 37 kD fragment of UreB is sufficient to confer protection against Helicobacter felis infection in mice”). Finally, Thomas et al. (supra) showed that oral immunization of mice with crude sonicates of H. pylori protected mice from subsequent challenge with H. felis.

Polynucleotides, e.g., DNA molecules, encoding allelic variants can easily be obtained by polymerase chain reaction (PCR) amplification of genomic bacterial DNA extracted by conventional methods. This involves the use of synthetic oligonucleotide primers matching sequences that are upstream and downstream of the 5′ and 3′ ends of the coding region. Suitable primers can be designed based on the nucleotide sequence information provided in the sequence listing (odd numbers, up to SEQ ID NO: 629). Typically, a primer consists of 10 to 40, preferably 15 to 25 nucleotides. It can also be advantageous to select primers containing C and G nucleotides in proportions sufficient to ensure efficient hybridization, e.g., an amount of C and G nucleotides of at least 40%, preferably 50%, of the total nucleotide amount. Those skilled in the art can readily design primers that can be used to isolate the polynucleotides of the invention from different Helicobacter strains. Experimental conditions for carrying out PCR can readily be determined by one skilled in the art and an illustration of carrying out PCR is provided in Example 2. As is well known in the art, restriction endonuclease recognition sites that contain, typically, 4 to 6 nucleotides (for example, the sequences 5′-GGATCC-3′ (BamHI) or 5′-CTCGAG-3′ (XhoI)), can be included on the 5′ ends of the primers. Restriction sites can be selected by those skilled in the art so that the amplified DNA can be conveniently cloned into an appropriately digested vector, such as a plasmid.

Useful homologs that do not occur naturally can be designed using known methods for identifying regions of an antigen that are likely to be tolerant of amino acid sequence changes and/or deletions. For example, sequences of the antigen from different species can be compared to identify conserved sequences.

Polypeptide derivatives that are encoded by polynucleotides of the invention include, e.g., fragments, polypeptides having large internal deletions derived from full-length polypeptides, and fusion proteins. Polypeptide fragments of the invention can be derived from a polypeptide having a sequence homologous to any of the sequences of the sequence listing (even numbers, up to SEQ ID NO: 630), to the extent that the fragments retain the substantial antigenicity of the parent polypeptide (specific antigenicity). Polypeptide derivatives can also be constructed by large internal deletions that remove a substantial part of the parent polypeptide, while retaining specific antigenicity. Generally, polypeptide derivatives should be about at least 12 amino acids in length to maintain antigenicity. Advantageously, they can be at least 20 amino acids, preferably at least 50 amino acids, more preferably at least 75 amino acids, and most preferably at least 100 amino acids in length.

Useful polypeptide derivatives, e.g., polypeptide fragments, can be designed using computer-assisted analysis of amino acid sequences in order to identify sites in protein antigens having potential as surface-exposed, antigenic regions (Hughes et al., Infect. Immun. 60(9):3497, 1992). For example, the Laser Gene Program from DNA Star can be used to obtain hydrophilicity, antigenic index, and intensity index plots for the polypeptides of the invention. This program can also be used to obtain information about homologies of the polypeptides with known protein motifs. One skilled in the art can readily use the information provided in such plots to select peptide fragments for use as vaccine antigens. For example, fragments spanning regions of the plots in which the antigenic index is relatively high can be selected. One can also select fragments spanning regions in which both the antigenic index and the intensity plots are relatively high. Fragments containing conserved sequences, particularly hydrophilic conserved sequences, can also be selected.

Polypeptide fragments and polypeptides having large internal deletions can be used for revealing epitopes that are otherwise masked in the parent polypeptide and that may be of importance for inducing a protective T cell-dependent immune response. Deletions can also remove immunodominant regions of high variability among strains.

It is an accepted practice in the field of immunology to use fragments and variants of protein immunogens as vaccines, as all that is required to induce an immune response to a protein is a small (e.g., 8 to 10 amino acids) immunogenic region of the protein. This has been done for a number of vaccines against pathogens other than Helicobacter. For example, short synthetic peptides corresponding to surface-exposed antigens of pathogens such as murine mammary tumor virus (peptide containing 11 amino acids; Dion et al., Virology 179:474-477, 1990), Semliki Forest virus (peptide containing 16 amino acids; Snijders et al., J. Gen. Virol. 72:557-565, 1991), and canine parvovirus (2 overlapping peptides, each containing 15 amino acids; Langeveld et al., Vaccine 12(15):1473-1480, 1994) have been shown to be effective vaccine antigens against their respective pathogens.

Polynucleotides encoding polypeptide fragments and polypeptides having large internal deletions can be constructed using standard methods (see, e.g., Ausubel et al., Current Protocols in Molecular Biology, John Wiley & Sons Inc., 1994), for example, by PCR, including inverse PCR, by restriction enzyme treatment of the cloned DNA molecules, or by the method of Kunkel et al. (Proc. Natl. Acad. Sci. U.S.A. 82:448, 1985; biological material available at Stratagene).

A polypeptide derivative can also be produced as a fusion polypeptide that contains a polypeptide or a polypeptide derivative of the invention fused, e.g., at the N- or C-terminal end, to any other polypeptide (hereinafter referred to as a peptide tail). Such a product can be easily obtained by translation of a genetic fusion, i.e., a hybrid gene. Vectors for expressing fusion polypeptides are commercially available, and include the pMal-c2 or pMal-p2 systems of New England Biolabs, in which the peptide tail is a maltose binding protein, the glutathione-S-transferase system of Pharmacia, or the His-Tag system available from Novagen. These and other expression systems provide convenient means for further purification of polypeptides and derivatives of the invention.

Another particular example of fusion polypeptides included in invention includes a polypeptide or polypeptide derivative of the invention fused to a polypeptide having adjuvant activity, such as, e.g., subunit B of either cholera toxin or E. coli heat-labile toxin. Several possibilities can be used for producing such fusion proteins. First, the polypeptide of the invention can be fused to the N-terminal end or, preferably, to the C-terminal end of the polypeptide having adjuvant activity. Second, a polypeptide fragment of the invention can be fused within the amino acid sequence of the polypeptide having adjuvant activity. Spacer sequences can also be included, if desired.

As stated above, the polynucleotides of the invention encode Helicobacter polypeptides in precursor or mature form. They can also encode hybrid precursors containing heterologous signal peptides, which can mature into polypeptides of the invention. By “heterologous signal peptide” is meant a signal peptide that is not found in the naturally-occurring precursor of a polypeptide of the invention.

A polynucleotide of the invention hybridizes, preferably under stringent conditions, to a polynucleotide having a sequence as shown in the sequence listing (odd numbers, up to SEQ ID NO: 629). Hybridization procedures are, e.g., described by Ausubel et al. (supra); Silhavy et al. (Experiments with Gene Fusions, Cold Spring Harbor Laboratory Press, Cold Spring Harbor, N.Y., 1984); and Davis et al. (A Manual for Genetic Engineering: Advanced Bacterial Genetics, Cold Spring Harbor Laboratory Press, Cold Spring Harbor, N.Y., 1980). Important parameters that can be considered for optimizing hybridization conditions are reflected in the following formula, which facilitates calculation of the melting temperature (Tm), which is the temperature above which two complementary DNA strands separate from one another (Casey et al., Nucl. Acid Res. 4:1539, 1977): Tm=81.5+0.5×(% G+C)+1.6 log (positive ion concentration)−0.6×(% formamide). Under appropriate stringency conditions, hybridization temperature (Th) is approximately 20 to 40° C., 20 to 25° C., or, preferably, 30 to 40° C. below the calculated Tm. Those skilled in the art will understand that optimal temperature and salt conditions can be readily determined empirically in preliminary experiments using conventional procedures. For example, stringent conditions can be achieved, both for pre-hybridizing and hybridizing incubations, (i) within 4-16 hours at 42° C., in 6×SSC containing 50% formamide or (ii) within 4-16 hours at 65° C. in an aqueous 6×SSC solution (1 M NaCl, 0.1 M sodium citrate (pH 7.0)). For polynucleotides containing 30 to 600 nucleotides, the above formula is used and then is corrected by subtracting (600/polynucleotide size in base pairs). Stringency conditions are defined by a Th that is 5 to 10° C. below Tm.

Hybridization conditions with oligonucleotides shorter than 20-30 bases do not precisely follow the rules set forth above. In such cases, the formula for calculating the Tm is as follows: Tm=4×(G+C)+2(A+T). For example, an 18 nucleotide fragment of 50% G+C would have an approximate Tm of 54° C.

A polynucleotide molecule of the invention, containing RNA, DNA, or modifications or combinations thereof, can have various applications. For example, a polynucleotide molecule can be used (i) in a process for producing the encoded polypeptide in a recombinant host system, (ii) in the construction of vaccine vectors such as poxviruses, which are further used in methods and compositions for preventing and/or treating Helicobacter infection, (iii) as a vaccine agent, in a naked form or formulated with a delivery vehicle and, (iv) in the construction of attenuated Helicobacter strains that can over-express a polynucleotide of the invention or express it in a non-toxic, mutated form.

According to a second aspect of the invention, there is therefore provided (i) an expression cassette containing a polynucleotide molecule of the invention placed under the control of elements (e.g., a promoter) required for expression; (ii) an expression vector containing an expression cassette of the invention; (iii) a procaryotic or eucaryotic cell transformed or transfected with an expression cassette and/or vector of the invention, as well as (iv) a process for producing a polypeptide or polypeptide derivative encoded by a polynucleotide of the invention, which involves culturing a procaryotic or eucaryotic cell transformed or transfected with an expression cassette and/or vector of the invention, under conditions that allow expression of the polynucleotide molecule of the invention and, recovering the encoded polypeptide or polypeptide derivative from the cell culture.

A recombinant expression system can be selected from procaryotic and eucaryotic hosts. Eucaryotic hosts include, for example, yeast cells (e.g., Saccharomyces cerevisiae or Pichia pastoris), mammalian cells (e.g., COS1, NIH3T3, or JEG3 cells), arthropods cells (e.g., Spodoptera frugiperda (SF9) cells), and plant cells. Preferably, a procaryotic host such as E. coli is used. Bacterial and eucaryotic cells are available from a number of different sources that are known to those skilled in the art, e.g., the American Type Culture Collection (ATCC; Rockville, Md.).

The choice of the expression cassette will depend on the host system selected, as well as the features desired for the expressed polypeptide. For example, it may be useful to produce a polypeptide of the invention in a particular lipidated form or any other form. Typically, an expression cassette includes a constitutive or inducible promoter that is functional in the selected host system; a ribosome binding site; a start codon (ATG); if necessary, a region encoding a signal peptide, e.g., a lipidation signal peptide; a polynucleotide molecule of the invention; a stop codon; and, optionally, a 3′ terminal region (translation and/or transcription terminator). The signal peptide-encoding region is adjacent to the polynucleotide of the invention and is placed in the proper reading frame. The signal peptide-encoding region can be homologous or heterologous to the polynucleotide molecule encoding the mature polypeptide and it can be specific to the secretion apparatus of the host used for expression. The open reading frame constituted by the polynucleotide molecule of the invention, alone or together with the signal peptide, is placed under the control of the promoter so that transcription and translation occur in the host system. Promoters and signal peptide-encoding regions are widely known and available to those skilled in the art and include, for example, the promoter of Salmonella typhimurium (and derivatives) that is inducible by arabinose (promoter araB) and is functional in Gram-negative bacteria such as E. coli (U.S. Pat. No. 5,028,530; Cagnon et al., Protein Engineering 4(7):843, 1991); the promoter of the bacteriophage T7 RNA polymerase gene, which is functional in a number of E. coli strains expressing T7 polymerase (U.S. Pat. No. 4,952,496); the OspA lipidation signal peptide; and RlpB lipidation signal peptide (Takase et al., J. Bact. 169:5692, 1987).

The expression cassette is typically part of an expression vector, which is selected for its ability to replicate in the chosen expression system. Expression vectors (e.g., plasmids or viral vectors) can be chosen from, for example, those described in Pouwels et al. (Cloning Vectors: A Laboratory Manual, 1985, Supp. 1987) and can purchased from various commercial sources. Methods for transforming or transfecting host cells with expression vectors are well known in the art and will depend on the host system selected, as described in Ausubel et al. (supra).

Upon expression, a recombinant polypeptide of the invention (or a polypeptide derivative) is produced and remains in the intracellular compartment, is secreted/excreted in the extracellular medium or in the periplasmic space, or is embedded in the cellular membrane. The polypeptide can then be recovered in a substantially purified form from the cell extract or from the supernatant after centrifugation of the cell culture. Typically, the recombinant polypeptide can be purified by antibody-based affinity purification or by any other method known to a person skilled in the art, such as by genetic fusion to a small affinity-binding domain. Antibody-based affinity purification methods are also available for purifying a polypeptide of the invention extracted from a Helicobacter strain. Antibodies useful for immunoaffinity purification of the polypeptides of the invention can be obtained using methods described below.

Polynucleotides of the invention can also be used in DNA vaccination methods, using either a viral or bacterial host as gene delivery vehicle (live vaccine vector) or administering the gene in a free form, e.g., inserted into a plasmid. Therapeutic or prophylactic efficacy of a polynucleotide of the invention can be evaluated as is described below.

Accordingly, in a third aspect of the invention, there is provided (i) a vaccine vector such as a poxvirus, containing a polynucleotide molecule of the invention placed under the control of elements required for expression; (ii) a composition of matter containing a vaccine vector of the invention, together with a diluent or carrier; (iii) a pharmaceutical composition containing a therapeutically or prophylactically effective amount of a vaccine vector of the invention; (iv) a method for inducing an immune response against Helicobacter in a mammal (e.g., a human; alternatively, the method can be used in veterinary applications for treating or preventing Helicobacter infection of animals, e.g., cats or birds), which involves administering to the mammal an immunogenically effective amount of a vaccine vector of the invention to elicit an immune response, e.g., a protective or therapeutic immune response to Helicobacter; and (v) a method for preventing and/or treating a Helicobacter (e.g., H. pylori, H. felis, H. mustelae, or H. heilmanii) infection, which involves administering a prophylactic or therapeutic amount of a vaccine vector of the invention to an individual in need. Additionally, the third aspect of the invention encompasses the use of a vaccine vector of the invention in the preparation of a medicament for preventing and/or treating Helicobacter infection.

A vaccine vector of the invention can express one or several polypeptides or derivatives of the invention, as well as at least one additional Helicobacter antigen such as a urease apoenzyme or a subunit, fragment, homolog, mutant, or derivative thereof. In addition, it can express a cytokine, such as interleukin-2 (IL-2) or interleukin-12 (IL-12), that enhances the immune response. Thus, a vaccine vector can include an additional polynucleotide molecules encoding, e.g., urease subunit A, B, or both, or a cytokine, placed under the control of elements required for expression in a mammalian cell.

Alternatively, a composition of the invention can include several vaccine vectors, each of which being capable of expressing a polypeptide or derivative of the invention. A composition can also contain a vaccine vector capable of expressing an additional Helicobacter antigen such as urease apoenzyme, a subunit, fragment, homolog, mutant, or derivative thereof, or a cytokine such as IL-2 or IL-12.

In vaccination methods for treating or preventing infection in a mammal, a vaccine vector of the invention can be administered by any conventional route in use in the vaccine field, for example, to a mucosal (e.g., ocular, intranasal, oral, gastric, pulmonary, intestinal, rectal, vaginal, or urinary tract) surface or via a parenteral (e.g., subcutaneous, intradermal, intramuscular, intravenous, or intraperitoneal) route. Preferred routes depend upon the choice of the vaccine vector. The administration can be achieved in a single dose or repeated at intervals. The appropriate dosage depends on various parameters that are understood by those skilled in the art, such as the nature of the vaccine vector itself, the route of administration, and the condition of the mammal to be vaccinated (e.g., the weight, age, and general health of the mammal).

Live vaccine vectors that can be used in the invention include viral vectors, such as adenoviruses and poxviruses, as well as bacterial vectors, e.g., Shigella, Salmonella, Vibrio cholerae, Lactobacillus, Bacille bilié de Calmette-Guérin (BCG), and Streptococcus. An example of an adenovirus vector, as well as a method for constructing an adenovirus vector capable of expressing a polynucleotide molecule of the invention, is described in U.S. Pat. No. 4,920,209. Poxvirus vectors that can be used in the invention include, e.g., vaccinia and canary pox viruses, which are described in U.S. Pat. No. 4,722,848 and U.S. Pat. No. 5,364,773, respectively (also see, e.g., Tartaglia et al., Virology 188:217, 1992, for a description of a vaccinia virus vector, and Taylor et al, Vaccine 13:539, 1995, for a description of a canary poxvirus vector). Poxvirus vectors capable of expressing a polynucleotide of the invention can be obtained by homologous recombination, as described in Kieny et al. (Nature 312:163, 1984) so that the polynucleotide of the invention is inserted in the viral genome under appropriate conditions for expression in mammalian cells. Generally, the dose of viral vector vaccine, for therapeutic or prophylactic use, can be from about 1×10⁴ to about 1×10¹¹, advantageously from about 1×10⁷ to about 1×10¹⁰, or, preferably, from about 1×10⁷ to about 1×10⁹ plaque-forming units per kilogram. Preferably, viral vectors are administered parenterally, for example, in 3 doses that are 4 weeks apart. Those skilled in the art will recognize that it is preferable to avoid adding a chemical adjuvant to a composition containing a viral vector of the invention and thereby minimizing the immune response to the viral vector itself.

Non-toxicogenic Vibrio cholerae mutant strains that can be used in live oral vaccines are described by Mekalanos et al. (Nature 306:551, 1983) and in U.S. Pat. No. 4,882,278 (strain in which a substantial amount of the coding sequence of each of the two ctxA alleles has been deleted so that no functional cholerae toxin is produced); WO 92/11354 (strain in which the irgA locus is inactivated by mutation; this mutation can be combined in a single strain with ctxA mutations); and WO 94/1533 (deletion mutant lacking functional ctxA and attRS1 DNA sequences). These strains can be genetically engineered to express heterologous antigens, as described in WO 94/19482. An effective vaccine dose of a V. cholerae strain capable of expressing a polypeptide or polypeptide derivative encoded by a polynucleotide molecule of the invention can contain, e.g., about 1×10⁵ to about 1×10⁹, preferably about 1×10⁶ to about 1×10⁸ viable bacteria in an appropriate volume for the selected route of administration. Preferred routes of administration include all mucosal routes, but, most preferably, these vectors are administered intranasally or orally.

Attenuated Salmonella typhimurium strains, genetically engineered for recombinant expression of heterologous antigens, and their use as oral vaccines, are described by Nakayama et al. (Bio/Technology 6:693, 1988) and in WO 92/11361. Preferred routes of administration for these vectors include all mucosal routes. Most preferably, the vectors are administered intranasally or orally.

Others bacterial strains useful as vaccine vectors are described by High et al. (EMBO 11:1991, 1992) and Sizemore et al. (Science 270:299, 1995; Shigella flexneri); Medaglini et al. (Proc. Natl. Acad. Sci. U.S.A. 92:6868, 1995; (Streptococcus gordonii); Flynn (Cell. Mol. Biol. 40 (suppl. I):31, 1194), and in WO 88/6626, WO 90/0594, WO 91/13157, WO 92/1796, and WO 92/21376 (Bacille Calmette Guerin). In bacterial vectors, a polynucleotide of the invention can be inserted into the bacterial genome or it can remain in a free state, for example, carried on a plasmid.

An adjuvant can also be added to a composition containing a bacterial vector vaccine. A number of adjuvants that can be used are known to those skilled in the art. For example, preferred adjuvants can be selected from the list provided below.

According to a fourth aspect of the invention, there is also provided (i) a composition of matter containing a polynucleotide of the invention, together with a diluent or carrier; (ii) a pharmaceutical composition containing a therapeutically or prophylactically effective amount of a polynucleotide of the invention; (iii) a method for inducing an immune response against Helicobacter, in a mammal, by administering to the mammal an immunogenically effective amount of a polynucleotide of the invention to elicit an immune response, e.g., a protective immune response to Helicobacter; and (iv) a method for preventing and/or treating a Helicobacter (e.g., H. pylori, H. felis, H. mustelae, or H. heilmanii) infection, by administering a prophylactic or therapeutic amount of a polynucleotide of the invention to an individual in need of such treatment. Additionally, the fourth aspect of the invention encompasses the use of a polynucleotide of the invention in the preparation of a medicament for preventing and/or treating Helicobacter infection. The fourth aspect of the invention preferably includes the use of a polynucleotide molecule placed under conditions for expression in a mammalian cell, e.g., in a plasmid that is unable to replicate in mammalian cells and to substantially integrate into a mammalian genome.

Polynucleotides (for example, DNA or RNA molecules) of the invention can also be administered as such to a mammal as a vaccine. When a DNA molecule of the invention is used, it can be in the form of a plasmid that is unable to replicate in a mammalian cell and unable to integrate into the mammalian genome. Typically, a DNA molecule is placed under the control of a promoter suitable for expression in a mammalian cell. The promoter can function ubiquitously or tissue-specifically. Examples of non-tissue specific promoters include the early Cytomegalovirus (CMV) promoter (U.S. Pat. No. 4,168,062) and the Rous Sarcoma Virus promoter (Norton et al., Molec. Cell Biol. 5:281, 1985). The desmin promoter (Li et al., Gene 78:243, 1989; Li et al., J. Biol. Chem. 266:6562, 1991; Li et al., J. Biol. Chem. 268:10403, 1993) is tissue-specific and drives expression in muscle cells. More generally, useful promoters and vectors are described, e.g., in WO 94/21797 and by Hartikka et al. (Human Gene Therapy 7:1205, 1996).

For DNA/RNA vaccination, the polynucleotide of the invention can encode a precursor or a mature form of a polypeptide of the invention. When it encodes a precursor form, the precursor sequence can be homologous or heterologous. In the latter case, a eucaryotic leader sequence can be used, such as the leader sequence of the tissue-type plasminogen factor (tPA).

A composition of the invention can contain one or several polynucleotides of the invention. It can also contain at least one additional polynucleotide encoding another Helicobacter antigen, such as urease subunit A, B, or both, or a fragment, derivative, mutant, or analog thereof. A polynucleotide encoding a cytokine, such as interleukin-2 (IL-2) or interleukin-12 (IL-12), can also be added to the composition so that the immune response is enhanced. These additional polynucleotides are placed under appropriate control for expression. Advantageously, DNA molecules of the invention and/or additional DNA molecules to be included in the same composition are carried in the same plasmid.

Standard methods can be used in the preparation of therapeutic polynucleotides of the invention. For example, a polynucleotide can be used in a naked form, free of any delivery vehicles, such as anionic liposomes, cationic lipids, microparticles, e.g., gold microparticles, precipitating agents, e.g., calcium phosphate, or any other transfection-facilitating agent. In this case, the polynucleotide can be simply diluted in a physiologically acceptable solution, such as sterile saline or sterile buffered saline, with or without a carrier. When present, the carrier preferably is isotonic, hypotonic, or weakly hypertonic, and has a relatively low ionic strength, such as provided by a sucrose solution, e.g., a solution containing 20% sucrose.

Alternatively, a polynucleotide can be associated with agents that assist in cellular uptake. It can be, e.g., (i) complemented with a chemical agent that modifies cellular permeability, such as bupivacaine (see, e.g., WO 94/16737), (ii) encapsulated into liposomes, or (iii) associated with cationic lipids or silica, gold, or tungsten microparticles.

Anionic and neutral liposomes are well-known in the art (see, e.g., Liposomes: A Practical Approach, RPC New Ed, IRL Press, 1990, for a detailed description of methods for making liposomes) and are useful for delivering a large range of products, including polynucleotides.

Cationic lipids can also be used for gene delivery. Such lipids include, for example, Lipofectin™, which is also known as DOTMA (N-[1-(2,3-dioleyloxy)propyl]-N,N,N-trimethylammonium chloride), DOTAP (1,2-bis(oleyloxy)-3-(trimethylammonio)propane), DDAB (dimethyldioctadecylammonium bromide), DOGS (dioctadecylamidologlycyl spermine), and cholesterol derivatives. A description of these cationic lipids can be found in EP 187,702, WO 90/11092, U.S. Pat. No. 5,283,185, WO 91/15501, WO 95/26356, and U.S. Pat. No. 5,527,928. Cationic lipids for gene delivery are preferably used in association with a neutral lipid such as DOPE (dioleyl phosphatidylethanolamine; WO 90/11092). Other transfection-facilitating compounds can be added to a formulation containing cationic liposomes. A number of them are described in, e.g., WO 93/18759, WO 93/19768, WO 94/25608, and WO 95/2397. They include, e.g., spermine derivatives useful for facilitating the transport of DNA through the nuclear membrane (see, for example, WO 93/18759) and membrane-permeabilizing compounds such as GALA, Gramicidine S, and cationic bile salts (see, for example, WO 93/19768).

Gold or tungsten microparticles can also be used for gene delivery, as described in WO 91/359, WO 93/17706, and by Tang et al. (Nature 356:152, 1992). In this case, the microparticle-coated polynucleotides can be injected via intradermal or intraepidermal routes using a needleless injection device (“gene gun”), such as those described in U.S. Pat. No. 4,945,050, U.S. Pat. No. 5,015,580, and WO 94/24263.

The amount of DNA to be used in a vaccine recipient depends, e.g., on the strength of the promoter used in the DNA construct, the immunogenicity of the expressed gene product, the condition of the mammal intended for administration (e.g., the weight, age, and general health of the mammal), the mode of administration, and the type of formulation. In general, a therapeutically or prophylactically effective dose from about 1 μg to about 1 mg, preferably, from about 10 μg to about 800 μg, and, more preferably, from about 25 μg to about 250 μg, can be administered to human adults. The administration can be achieved in a single dose or repeated at intervals.

The route of administration can be any conventional route used in the vaccine field. As general guidance, a polynucleotide of the invention can be administered via a mucosal surface, e.g., an ocular, intranasal, pulmonary, oral, intestinal, rectal, vaginal, or urinary tract surface, or via a parenteral route, e.g., by an intravenous, subcutaneous, intraperitoneal, intradermal, intraepidermal, or intramuscular route. The choice of administration route will depend on, e.g., the formulation that is selected. A polynucleotide formulated in association with bupivacaine is advantageously administered into muscle. When a neutral or anionic liposome or a cationic lipid, such as DOTMA, is used, the formulation can be advantageously injected via intravenous, intranasal (for example, by aerosolization), intramuscular, intradermal, and subcutaneous routes. A polynucleotide in a naked form can advantageously be administered via the intramuscular, intradermal, or subcutaneous routes. Although not absolutely required, such a composition can also contain an adjuvant. A systemic adjuvant that does not require concomitant administration in order to exhibit an adjuvant effect is preferable.

The sequence information provided in the present application enables the design of specific nucleotide probes and primers that can be used in diagnostic methods. Accordingly, in a fifth aspect of the invention, there is provided a nucleotide probe or primer having a sequence found in, or derived by degeneracy of the genetic code from, a sequence shown in the sequence listing (odd numbers, up to SEQ ID NO: 629).

The term “probe” as used in the present application refers to DNA (preferably single stranded) or RNA molecules (or modifications or combinations thereof) that hybridize under the stringent conditions, as defined above, to polynucleotide molecules having sequences homologous to any of those shown in the sequence listing (odd numbers, up to SEQ ID NO: 629), or to a complementary or anti-sense sequence of any of those shown in the sequence listing (odd numbers, up to SEQ ID NO: 629). Generally, probes are significantly shorter than the full-length sequences shown in the sequence listing. For example, they can contain from about 5 to about 100, preferably from about 10 to about 80 nucleotides. In particular, probes have sequences that are at least 75%, preferably at least 85%, more preferably 95% homologous to a portion of a sequence as shown in the sequence listing (odd numbers, up to SEQ ID NO: 629), or a sequence complementary to any of such sequences.

Probes can contain modified bases, such as inosine, methyl-5-deoxycytidine, deoxyuridine, dimethylamino-5-deoxyuridine, or diamino-2,6-purine. Sugar or phosphate residues can also be modified or substituted. For example, a deoxyribose residue can be replaced by a polyamide (Nielsen et al., Science 254:1497, 1991) and phosphate residues can be replaced by ester groups such as diphosphate, alkyl, arylphosphonate, and phosphorothioate esters. In addition, the 2′-hydroxyl group on ribonucleotides can be modified by addition of, e.g., alkyl groups.

Probes of the invention can be used in diagnostic tests, or as capture or detection probes. Such capture probes can be immobilized on solid supports, directly or indirectly, by covalent means or by passive adsorption. A detection probe can be labeled by a detectable label, for example a label selected from radioactive isotopes; enzymes, such as peroxidase and alkaline phosphatase; enzymes that are able to hydrolyze a chromogenic, fluorogenic, or luminescent substrate; compounds that are chromogenic, fluorogenic, or luminescent; nucleotide base analogs; and biotin.

Probes of the invention can be used in any conventional hybridization method, such as in dot blot methods (Maniatis et al., Molecular Cloning: A Laboratory Manual, Cold Spring Harbor Laboratory Press, Cold Spring Harbor, N.Y., 1982), Southern blot methods (Southern, J. Mol. Biol. 98:503, 1975), northern blot methods (identical to Southern blot to the exception that RNA is used as a target), or a sandwich method (Dunn et al., Cell 12:23, 1977). As is known in the art, the latter technique involves the use of a specific capture probe and a specific detection probe that have nucleotide sequences that are at least partially different from each other.

Primers used in the invention usually contain about 10 to 40 nucleotides and are used to initiate enzymatic polymerization of DNA in an amplification process (e.g., PCR), an elongation process, or a reverse transcription method. In a diagnostic method involving PCR, the primers can be labeled.

Thus, the invention also encompasses (i) a reagent containing a probe of the invention for detecting and/or identifying the presence of Helicobacter in a biological material; (ii) a method for detecting and/or identifying the presence of Helicobacter in a biological material, in which (a) a sample is recovered or derived from the biological material, (b) DNA or RNA is extracted from the material and denatured, and (c) the sample is exposed to a probe of the invention, for example, a capture probe, a detection probe, or both, under stringent hybridization conditions, so that hybridization is detected; and (iii) a method for detecting and/or identifying the presence of Helicobacter in a biological material, in which (a) a sample is recovered or derived from the biological material, (b) DNA is extracted therefrom, (c) the extracted DNA is contacted with at least one, or, preferably two, primers of the invention, and amplified by the polymerase chain reaction, and (d) an amplified DNA molecule is produced.

As mentioned above, polypeptides that can be produced by expression of the polynucleotides of the invention can be used as vaccine antigens. Accordingly, a sixth aspect of the invention features a substantially purified polypeptide or polypeptide derivative having an amino acid sequence encoded by a polynucleotide of the invention.

A “substantially purified polypeptide” is defined as a polypeptide that is separated from the environment in which it naturally occurs and/or a polypeptide that is free of most of the other polypeptides that are present in the environment in which it was synthesized. The polypeptides of the invention can be purified from a natural source, such as a Helicobacter strain, or can be produced using recombinant methods.

Homologous polypeptides or polypeptide derivatives encoded by polynucleotides of the invention can be screened for specific antigenicity by testing cross-reactivity with an antiserum raised against a polypeptide having an amino acid sequence as shown in the sequence listing (even numbers, up to SEQ ID NO: 630). Briefly, a monospecific hyperimmune antiserum can be raised against a purified reference polypeptide as such or as a fusion polypeptide, for example, an expression product of MBP, GST, or His-tag systems, or a synthetic peptide predicted to be antigenic. The homologous polypeptide or derivative that is screened for specific antigenicity can be produced as such or as a fusion polypeptide. In the latter case, and if the antiserum is also raised against a fusion polypeptide, two different fusion systems are employed. Specific antigenicity can be determined using a number of methods, including Western blot (Towbin et al., Proc. Natl. Acad. Sci. U.S.A. 76:4350, 1979), dot blot, and ELISA methods, as described below.

In a Western blot assay, the product to be screened, either as a purified preparation or a total E. coli extract, is fractionated by SDS-PAGE, as described, for example, by Laemmli (Nature 227:680, 1970). After being transferred to a filter, such as a nitrocellulose membrane, the material is incubated with the mono specific hyperimmune antiserum, which is diluted in a range of dilutions from about 1:50 to about 1:5000, preferably from about 1:100 to about 1:500. Specific antigenicity is shown once a band corresponding to the product exhibits reactivity at any of the dilutions in the range.

In an ELISA assay, the product to be screened can be used as the coating antigen. A purified preparation is preferred, but a whole cell extract can also be used. Briefly, about 100 μL of a preparation of about 10 μg protein/ml is distributed into wells of a 96-well ELISA plate. The plate is incubated for about 2 hours at 37° C., then overnight at 4° C. The plate is washed with phosphate buffer saline (PBS) containing 0.05% Tween 20 (PBS/Tween buffer) and the wells are saturated with 250 μL PBS containing 1% bovine serum albumin (BSA), to prevent non-specific antibody binding. After 1 hour of incubation at 37° C., the plate is washed with PBS/Tween buffer. The antiserum is serially diluted in PBS/Tween buffer containing 0.5% BSA, and 100 μL dilutions are added to each well. The plate is incubated for 90 minutes at 37° C., washed, and evaluated using standard methods. For example, a goat anti-rabbit peroxidase conjugate can be added to the wells when the specific antibodies used were raised in rabbits. Incubation is carried out for about 90 minutes at 37° C. and the plate is washed. The reaction is developed with the appropriate substrate and the reaction is measured by colorimetry (absorbance measured spectrophotometrically). Under these experimental conditions, a positive reaction is shown once an O.D. value of 1.0 is detected with a dilution of at least about 1:50, preferably of at least about 1:500.

In a dot blot assay, a purified product is preferred, although a whole cell extract can be used. Briefly, a solution of the product at a concentration of about 100 μg/ml is serially diluted two-fold with 50 mM Tris-HCl (pH 7.5). One hundred μL of each dilution is applied to a filter, such as a 0.45 μm nitrocellulose membrane, set in a 96-well dot blot apparatus (Biorad). The buffer is removed by applying vacuum to the system. Wells are washed by addition of 50 mM Tris-HCl (pH 7.5) and the membrane is air-dried. The membrane is saturated in blocking buffer (50 mM Tris-HCl (pH 7.5), 0.15 M NaCl, 10 g/L skim milk) and incubated with an antiserum diluted from about 1:50 to about 1:5000, preferably about 1:500. The reaction is detected using standard methods. For example, a goat anti-rabbit peroxidase conjugate can be added to the wells when rabbit antibodies are used. Incubation is carried out for about 90 minutes at 37° C. and the blot is washed. The reaction is developed with the appropriate substrate and stopped. The reaction is then measured visually by the appearance of a colored spot, e.g., by colorimetry. Under these experimental conditions, a positive reaction is associated with detection of a colored spot for reactions carried out with a dilution of at least about 1:50, preferably, of at least about 1:500. Therapeutic or prophylactic efficacy of a polypeptide or polypeptide derivative of the invention can be evaluated as described below.

According to a seventh aspect of the invention, there is provided (i) a composition of matter containing a polypeptide of the invention together with a diluent or carrier; (ii) a pharmaceutical composition containing a therapeutically or prophylactically effective amount of a polypeptide of the invention; (iii) a method for inducing an immune response against Helicobacter in a mammal by administering to the mammal an immunogenically effective amount of a polypeptide of the invention to elicit an immune response, e.g., a protective immune response to Helicobacter; and (iv) a method for preventing and/or treating a Helicobacter (e.g., H. pylori, H. felis, H. mustelae, or H. heilmanii) infection, by administering a prophylactic or therapeutic amount of a polypeptide of the invention to an individual in need of such treatment. Additionally, this aspect of the invention includes the use of a polypeptide of the invention in the preparation of a medicament for preventing and/or treating Helicobacter infection.

The immunogenic compositions of the invention can be administered by any conventional route in use in the vaccine field, for example, to a mucosal (e.g., ocular, intranasal, pulmonary, oral, gastric, intestinal, rectal, vaginal, or urinary tract) surface or via a parenteral (e.g., subcutaneous, intradermal, intramuscular, intravenous, or intraperitoneal) route. The choice of the administration route depends upon a number of parameters, such as the adjuvant used. For example, if a mucosal adjuvant is used, the intranasal or oral route will be preferred, and if a lipid formulation or an aluminum compound is used, a parenteral route will be preferred. In the latter case, the subcutaneous or intramuscular route is most preferred. The choice of administration route can also depend upon the nature of the vaccine agent. For example, a polypeptide of the invention fused to CTB or to LTB will be best administered to a mucosal surface.

A composition of the invention can contain one or several polypeptides or derivatives of the invention. It can also contain at least one additional Helicobacter antigen, such as the urease apoenzyme, or a subunit, fragment, homolog, mutant, or derivative thereof.

For use in a composition of the invention, a polypeptide or polypeptide derivative can be formulated into or with liposomes, such as neutral or anionic liposomes, microspheres, ISCOMS, or virus-like particles (VLPs), to facilitate delivery and/or enhance the immune response. These compounds are readily available to those skilled in the art; for example, see Liposomes: A Practical Approach (supra). Adjuvants other than liposomes can also be used in the invention and are well known in the art (see, for example, the list provided below).

Administration can be achieved in a single dose or repeated as necessary at intervals that can be determined by one skilled in the art. For example, a priming dose can be followed by three booster doses at weekly or monthly intervals. An appropriate dose depends on various parameters, including the nature of the recipient (e.g., whether the recipient is an adult or an infant), the particular vaccine antigen, the route and frequency of administration, the presence/absence or type of adjuvant, and the desired effect (e.g., protection and/or treatment), and can be readily determined by one skilled in the art. In general, a vaccine antigen of the invention can be administered mucosally in an amount ranging from about 10 μg to about 500 mg, preferably from about 1 mg to about 200 mg. For a parenteral route of administration, the dose usually should not exceed about 1 mg, and is, preferably, about 100 μg.

When used as components of a vaccine, the polynucleotides and polypeptides of the invention can be used sequentially as part of a multi-step immunization process. For example, a mammal can be initially primed with a vaccine vector of the invention, such as a pox virus, e.g., via a parenteral route, and then boosted twice with a polypeptide encoded by the vaccine vector, e.g., via the mucosal route. In another example, liposomes associated with a polypeptide or polypeptide derivative of the invention can be used for priming, with boosting being carried out mucosally using a soluble polypeptide or polypeptide derivative of the invention, in combination with a mucosal adjuvant (e.g., LT).

Polypeptides and polypeptide derivatives of the invention can also be used as diagnostic reagents for detecting the presence of anti-Helicobacter antibodies, e.g., in blood samples. Such polypeptides can be about 5 to about 80, preferably, about 10 to 15 about 50 amino acids in length and can be labeled or unlabeled, depending upon the diagnostic method. Diagnostic methods involving such a reagent are described below.

Upon expression of a polynucleotide molecule of the invention, a polypeptide or polypeptide derivative is produced and can be purified using known methods. For example, the polypeptide or polypeptide derivative can be produced as a fusion protein containing a fused tail that facilitates purification. The fusion product can be used to immunize a small mammal, e.g., a mouse or a rabbit, in order to raise monospecific antibodies against the polypeptide or polypeptide derivative. The eighth aspect of the invention thus provides a monospecific antibody that binds to a polypeptide or polypeptide derivative of the invention.

By “monospecific antibody” is meant an antibody that is capable of reacting with a unique, naturally-occurring Helicobacter polypeptide. An antibody of the invention can be polyclonal or monoclonal. Monospecific antibodies can be recombinant, e.g., chimeric (e.g., consisting of a variable region of murine origin and a human constant region), humanized (e.g., a human immunoglobulin constant region and a variable region of animal, e.g., murine, origin), and/or single chain. Both polyclonal and monospecific antibodies can also be in the form of immunoglobulin fragments, e.g., F(ab)′2 or Fab fragments. The antibodies of the invention can be of any isotype, e.g., IgG or IgA, and polyclonal antibodies can be of a single isotype or can contain a mixture of isotypes.

The antibodies of the invention, which can be raised to a polypeptide or polypeptide derivative of the invention, can be produced and identified using standard immunological assays, e.g., Western blot assays, dot blot assays, or ELISA (see, e.g., Coligan et al., Current Protocols in Immunology, John Wiley & Sons, Inc., New York, N.Y., 1994). The antibodies can be used in diagnostic methods to detect the presence of Helicobacter antigens in a sample, such as a biological sample. The antibodies can also be used in affinity chromatography methods for purifying a polypeptide or polypeptide derivative of the invention. As is discussed further below, the antibodies can also be used in prophylactic and therapeutic passive immunization methods.

Accordingly, a ninth aspect of the invention provides (i) a reagent for detecting the presence of Helicobacter in a biological sample that contains an antibody, polypeptide, or polypeptide derivative of the invention; and (ii) a diagnostic method for detecting the presence of Helicobacter in a biological sample, by contacting the biological sample with an antibody, a polypeptide, or a polypeptide derivative of the invention, so that an immune complex is formed, and detecting the complex as an indication of the presence of Helicobacter in the sample or the organism from which the sample was derived. The immune complex is formed between a component of the sample and the antibody, polypeptide, or polypeptide derivative, and that any unbound material can be removed prior to detecting the complex. A polypeptide reagent can be used for detecting the presence of anti-Helicobacter antibodies in a sample, e.g., a blood sample, while an antibody of the invention can be used for screening a sample, such as a gastric extract or biopsy sample, for the presence of Helicobacter polypeptides.

For use in diagnostic methods, the reagent (e.g., the antibody, polypeptide, or polypeptide derivative of the invention) can be in a free state or can be immobilized on a solid support, such as, for example, on the interior surface of a tube or on the surface, or within pores, of a bead. Immobilization can be achieved using direct or indirect means. Direct means include passive adsorption (i.e., non-covalent binding) or covalent binding between the support and the reagent. By “indirect means” is meant that an anti-reagent compound that interacts with the reagent is first attached to the solid support. For example, if a polypeptide reagent is used, an antibody that binds to it can serve as an anti-reagent, provided that it binds to an epitope that is not involved in recognition of antibodies in biological samples. Indirect means can also employ a ligand-receptor system, for example, a molecule, such as a vitamin, can be grafted onto the polypeptide reagent and the corresponding receptor can be immobilized on the solid phase. This concept is illustrated by the well known biotin-streptavidin system. Alternatively, indirect means can be used, e.g., by adding to the reagent a peptide tail, chemically or by genetic engineering, and immobilizing the grafted or fused product by passive adsorption or covalent linkage of the peptide tail.

According to a tenth aspect of the invention, there is provided a process for purifying, from a biological sample, a polypeptide or polypeptide derivative of the invention, which involves carrying out antibody-based affinity chromatography with the biological sample, wherein the antibody is a monospecific antibody of the invention.

For use in a purification process of the invention, the antibody can be polyclonal or monospecific, and preferably is of the IgG type. Purified IgGs can be prepared from an antiserum using standard methods (see, e.g., Coligan et al., supra). Conventional chromatography supports, as well as standard methods for grafting antibodies, are described, for example, by Harlow et al. (Antibodies: A Laboratory Manual, Cold Spring Harbor Laboratory Press, Cold Spring Harbor, N.Y., 1988).

Briefly, a biological sample, such as an H. pylori extract, preferably in a buffer solution, is applied to a chromatography material, which is, preferably, equilibrated with the buffer used to dilute the biological sample, so that the polypeptide or polypeptide derivative of the invention (i.e., the antigen) is allowed to adsorb onto the material. The chromatography material, such as a gel or a resin coupled to an antibody of the invention, can be in batch form or in a column. The unbound components are washed off and the antigen is eluted with an appropriate elution buffer, such as a glycine buffer, a buffer containing a chaotropic agent, e.g., guanidine HCl, or a buffer having high salt concentration (e.g., 3 M MgCl₂). Eluted fractions are recovered and the presence of the antigen is detected, e.g., by measuring the absorbance at 280 nm.

An antibody of the invention can be screened for therapeutic efficacy as follows. According to an eleventh aspect of the invention, there is provided (i) a composition of matter containing a monospecific antibody of the invention, together with a diluent or carrier; (ii) a pharmaceutical composition containing a therapeutically or prophylactically effective amount of a monospecific antibody of the invention, and (iii) a method for treating or preventing Helicobacter (e.g., H. pylori, H. felis, H. mustelae, or H. heilmanii) infection, by administering a therapeutic or prophylactic amount of a monospecific antibody of the invention to an individual in need of such treatment. In addition, the eleventh aspect of the invention includes the use of a monospecific antibody of the invention in the preparation of a medicament for treating or preventing Helicobacter infection.

The monospecific antibody can be polyclonal or monoclonal, and is, preferably, predominantly of the IgA isotype. In passive immunization methods, the antibody is administered to a mucosal surface of a mammal, e.g., the gastric mucosa, e.g., orally or intragastrically, optionally, in the presence of a bicarbonate buffer. Alternatively, systemic administration, not requiring a bicarbonate buffer, can be carried out. A monospecific antibody of the invention can be administered as a single active agent or as a mixture with at least one additional monospecific antibody specific for a different Helicobacter polypeptide. The amount of antibody and the particular regimen used can be readily determined by one skilled in the art. For example, daily administration of about 100 to 1,000 mg of antibody over one week, or three doses per day of about 100 to 1,000 mg of antibody over two or three days, can be effective regimens for most purposes.

Therapeutic or prophylactic efficacy can be evaluated using standard methods in the art, e.g., by measuring induction of a mucosal immune response or induction of protective and/or therapeutic immunity, using, e.g., the H. felis mouse model and the procedures described by Lee et al. (Eur. J. Gastroenterology & Hepatology 7:303, 1995) or Lee et al. (J. Infect. Dis. 172:161, 1995). Those skilled in the art will recognize that the H. felis strain of the model can be replaced with another Helicobacter strain. For example, the efficacy of polynucleotide molecules and polypeptides from H. pylori is, preferably, evaluated in a mouse model using an H. pylori strain. Protection can be determined by comparing the degree of Helicobacter infection in the gastric tissue assessed by, for example, urease activity, bacterial counts, or gastritis, to that of a control group. Protection is shown when infection is reduced by comparison to the control group. Such an evaluation can be made for polynucleotides, vaccine vectors, polypeptides, and polypeptide derivatives, as well as for antibodies of the invention.

For example, various doses of an antibody of the invention can be administered to the gastric mucosa of mice previously challenged with an H. pylori strain, as described, e.g., by Lee et al. (supra). Then, after an appropriate period of time, the bacterial load of the mucosa can be estimated by assessing urease activity, as compared to a control. Reduced urease activity indicates that the antibody is therapeutically effective.

Adjuvants that can be used in any of the vaccine compositions described above are described as follows. Adjuvants for parenteral administration include, for example, aluminum compounds, such as aluminum hydroxide, aluminum phosphate, and aluminum hydroxy phosphate. The antigen can be precipitated with, or adsorbed onto, the aluminum compound using standard methods. Other adjuvants, such as RIBI (ImmunoChem, Hamilton, Mont.), can also be used in parenteral administration.

Adjuvants that can be used for mucosal administration include, for example, bacterial toxins, e.g., the cholera toxin (CT), the E. coli heat-labile toxin (LT), the Clostridium difficile toxin A, the pertussis toxin (PT), and combinations, subunits, toxoids, or mutants thereof. For example, a purified preparation of native cholera toxin subunit B (CTB) can be used. Fragments, homologs, derivatives, and fusions to any of these toxins can also be used, provided that they retain adjuvant activity. Preferably, a mutant having reduced toxicity is used. Suitable mutants are described, e.g., in WO 95/17211 (Arg-7-Lys CT mutant), WO 96/6627 (Arg-192-Gly LT mutant), and WO 95/34323 (Arg-9-Lys and Glu-129-Gly PT mutant). Additional LT mutants that can be used in the methods and compositions of the invention include, e.g., Ser-63-Lys, Ala-69-Gly, Glu-110-Asp, and Glu-112-Asp mutants. Other adjuvants, such as the bacterial monophosphoryl lipid A (MPLA) of, e.g., E. coli, Salmonella minnesota, Salmonella typhimurium, or Shigella flexneri; saponins, and polylactide glycolide (PLGA) microspheres, can also be used in mucosal administration. Adjuvants useful for both mucosal and parenteral administrations, such as polyphosphazene (WO 95/2415), can also be used.

Any pharmaceutical composition of the invention, containing a polynucleotide, polypeptide, polypeptide derivative, or antibody of the invention, can be manufactured using standard methods. It can be formulated with a pharmaceutically acceptable diluent or carrier, e.g., water or a saline solution, such as phosphate buffer saline, optionally, including a bicarbonate salt, such as sodium bicarbonate, e.g., 0.1 to 0.5 M. Bicarbonate can advantageously be added to compositions intended for oral or intragastric administration. In general, a diluent or carrier can be selected on the basis of the mode and route of administration, and standard pharmaceutical practice. Suitable pharmaceutical carriers and diluents, as well as pharmaceutical necessities for their use in pharmaceutical formulations, are described in Remington's Pharmaceutical Sciences, a standard reference text in this field and in the USP/NF.

The invention also includes methods in which gastroduodenal infections, such as Helicobacter infection, are treated by oral administration of a Helicobacter polypeptide of the invention and a mucosal adjuvant, in combination with an antibiotic, an antisecretory agent, a bismuth salt, an antacid, sucralfate, or a combination thereof. Examples of such compounds that can be administered with the vaccine antigen and an adjuvant are antibiotics, including, e.g., macrolides, tetracyclines, β-lactams, aminoglycosides, quinolones, penicillins, and derivatives thereof (specific examples of antibiotics that can be used in the invention include, e.g., amoxicillin, clarithromycin, tetracycline, metronidizole, erythromycin, cefuroxime, and erythromycin); antisecretory agents, including, e.g., H₂-receptor antagonists (e.g., cimetidine, ranitidine, famotidine, nizatidine, and roxatidine), proton pump inhibitors (e.g., omeprazole, lansoprazole, and pantoprazole), prostaglandin analogs (e.g., misoprostil and enprostil), and anticholinergic agents (e.g., pirenzepine, telenzepine, carbenoxolone, and proglumide); and bismuth salts, including colloidal bismuth subcitrate, tripotassium dicitrate bismuthate, bismuth subsalicylate, bicitropeptide, and pepto-bismol (see, e.g., Goodwin et al., Helicobacter pylori, Biology and Clinical Practice, CRC Press, Boca Raton, Fla., pp 366-395, 1993; Physicians' Desk Reference, 49^{th} edn., Medical Economics Data Production Company, Montvale, N.J., 1995). In addition, compounds containing more than one of the above-listed components coupled together, e.g., ranitidine coupled to bismuth subcitrate, can be used. The invention also includes compositions for carrying out these methods, i.e., compositions containing a Helicobacter antigen (or antigens) of the invention, an adjuvant, and one or more of the above-listed compounds, in a pharmaceutically acceptable carrier or diluent.

Amounts of the above-listed compounds used in the methods and compositions of the invention can readily be determined by one skilled in the art. In addition, one skilled in the art can readily design treatment/immunization schedules. For example, the non-vaccine components can be administered on days 1-14, and the vaccine antigen+adjuvant can be administered on days 7, 14, 21, and 28.

Methods and pharmaceutical compositions of the invention can be used to treat or to prevent Helicobacter infections and, accordingly, gastroduodenal diseases associated with these infections, including acute, chronic, and atrophic gastritis, and peptic ulcer diseases, e.g., gastric and duodenal ulcers.

The invention is further illustrated by the following examples. Example 1 describes identification of genes, such as genes that encode the polypeptides of the invention, in the Helicobacter genome, as well as identification of signal sequences, and primer design for amplification of genes lacking signal sequences. Example 2 describes cloning of DNA molecules encoding polypeptides of the invention into a vector that provides a histidine tag, and production and purification of the resulting his-tagged fusion proteins. Example 3 describes methods for cloning DNA encoding the polypeptides of the invention so that they can be produced without his-tags, and Example 4 describes methods for purifying recombinantly produced polypeptides of the invention.

### EXAMPLE 1

### Identification of Genes in the H. pylori Genome, Identification of Signal Sequences, and Primer Design for Amplification of Genes Lacking Signal Sequences

1.A. Creating H. pylori Genomic Databases

The H. pylori genome was provided as a text file containing a single contiguous string of nucleotides that had been determined to be 1.76 Megabases in length. The complete genome was split into 17 separate files using the program SPLIT (Creativity in Action), giving rise to 16 contigs, each containing 100,000 nucleotides, and a 17^{th} contig containing the remaining 76,000 nucleotides. A header was added to each of the 17 files using the format: >hpg0.txt (representing contig 1), .hpg1.txt (representing contig 2), etc. The resulting 17 files, named hpg0 through hpg16, were then copied together to form one file that represented the plus strand of the complete H. pylori genome. The constructed database was given the designation “H.” A negative strand database of the H. pylori genome was created similarly by first creating a reverse complement of the positive strand using the program SeqPup (D. G. Gilbert, Indiana University Biology Department) and then performing the same procedure as described above for the plus strand. This database was given the designation “N.”

The regions predicted to encode open reading frames (ORFs) were defined for the complete H. pylori genome using the program GENEMARK™ (Borodovsky et al., Comp. Chem. 17:123, 1993). A database was created from a text file containing an annotated version of all ORFs predicted to be encoded by the H. pylori genome for both the plus and minus strands, and was given the designation “O.” Each ORF was assigned a number indicating its location on the genome and its position relative to other genes. No manipulation of the text file was required.

1.B. Searching the H. pylori Databases

The databases constructed as is described above were searched using the program FASTA (Pearson et al., Proc. Natl. Acad. Sci. U.S.A. 85:2444-2448, 1988). FASTA was used for searching either a DNA sequence against either of the gene databases (“H” and/or “N”), or a peptide sequence against the ORF library (“O”). TFASTX was used to search a peptide sequence against all possible reading frames of a DNA database (“H” and/or “N” libraries). Potential frameshifts also being resolved, FASTX was used for searching the translated reading frames of a DNA sequence against either a DNA database, or a peptide sequence against the protein database.

1.C. Isolation of DNA Sequences from the H. pylori Genome

The FASTA searches against the constructed DNA databases identified exact nucleotide coordinates on one or more of the isolated contigs, and therefore the location of the target DNA. Once the exact location of the target sequence was known, the contig identified to carry the gene was exported into the software package MapDraw (DNAStar, Inc.) and the gene was isolated. Gene sequences with flanking DNA was then excised and copied into the EditSeq. Software package (DNAStar, Inc.) for further analysis.

1.D. Identification of Signal Sequences

The deduced protein encoded by a target gene sequence is analyzed using the PROTEAN software package (DNAStar, Inc.). This analysis predicts those areas of the protein that are hydrophobic by using the Kyte-Doolittle algorithm, and identifies any potential polar residues preceding the hydrophobic core region, which is typical for many signal sequences. For confirmation, the target protein is then searched against a PROSITE database (DNAStar, Inc.) consisting of motifs and signatures. Characteristic of many signal sequences and hydrophobic regions in general, is the identification of predicted prokaryotic lipid attachment sites. Where confirmation between the two approaches is apparent at the N-terminus of any protein, putative cleavage sites are sought. Specifically, this includes the presence of either an Alanine (A), Serine (S), or Glycine (G) residue immediately after the core hydrophobic region. In the case of lipoproteins, a Cysteine (C) residue would be identified as the +1 residue, post-cleavage.

1.E. Rational Design of PCR Primers Based on the Identification of Signal Sequences

In order to clone gene sequences as N-terminus translational fusions for the generation of recombinant proteins with N-terminal Histidine tags, the gene sequence that specifies the signal sequence is omitted. The 5′-end of the gene-specific portion of the N-terminal primer is designed to start at the first codon beyond the cleavage site. In the case of lipoproteins, the 5′- end of the N-terminal primer begins at the second codon, immediately after the modifiable residue at position +1 post-cleavage. The omission of the signal sequence from the recombinant allows for one-step purification, and potential problems associated with insertion of signal sequences in the membrane of the host strain carrying the hybrid construct are avoided.

### EXAMPLE 2

### Preparation of Isolated DNA Encoding GHPO147, GHPO615, GHPO961, and GHPO1282, and Production of these Polypeptides as Histidine-Tagged Fusion Proteins

2.A. Preparation of Genomic DNA from Helicobacter pylori

H. pylori strain ORV2001, stored in LB medium containing 50% glycerol at −70° C., is grown on Colombia agar containing 7% sheep blood for 48 hours under microaerophilic conditions (8-10% CO₂, 5-7% O₂, 85-87% N₂). Cells are harvested, washed with phosphate buffer saline (PBS) (pH 7.2), and DNA is then extracted from the cells using the Rapid Prep Genomic DNA Isolation kit (Pharmacia Biotech).

2.B. PCR Amplification

DNA molecules encoding the polypeptides of the invention are amplified from genomic DNA, as can be prepared as is described above, by the Polymerase Chain Reaction (PCR) using primers that can readily be designed by one skilled in the art. For example, to amplify genes encoding GHPO147, GHPO615, GHPO961, and GHPO1282, the following primers can be used:

Appropriate amplification reaction conditions can readily be determined by one skilled in the art. For example, the following conditions can be used for amplification of DNA encoding GHPO615 using the primers set forth above: initial denaturation at 94° C. for 5 minutes, 25 cycles of denaturation at 97° C. for 30 seconds, hybridization at 55° C. for 1 minute, and elongation at 72° C. for 2 minutes, using Vent DNA polymerase. In the case of amplifying DNA encoding GHPO1282 with the primers set forth above, the following conditions can be used: initial denaturation at 94° C. for 5 minutes, 25 cycles of denaturation at 94° C. for 30 seconds, hybridization at 45° C. for 30 seconds, and elongation at 72° C. for 30 seconds, followed by a final elongation at 72° C. for 7 minutes, using Vent DNA polymerase.

2.C. Transformation and Selection of Transformants

A single PCR product is thus amplified and then is digested at 37° C. for 2 hours with BamHI and XhoI together in a 20 μl reaction volume. The digested product is ligated to similarly cleaved pET28a (Novagen) that is dephosphorylated prior to the ligation by treatment with Calf Intestinal Alkaline Phosphatase (CIP). The gene fusion constructed in this manner allows one-step affinity purification of the resulting fusion protein because of the presence of histidine residues at the N-terminus of the fusion protein, which are encoded by the vector.

The ligation reaction (20 μl) is carried out at 14° C. overnight and then is used to transform 100 μl fresh E. coli XL1-blue competent cells (Novagen). The cells are incubated on ice for 2 hours, heat-shocked at 42° C. for 30 seconds, and returned to ice for 90 seconds. The samples are then added to 1 ml LB broth in the absence of selection and grown at 37° C. for 2 hours. The cells are plated out on LB agar containing kanamycin (50 μg/ml) at a 10× and neat dilution and incubated overnight at 37 ° C. The following day, 50 colonies are picked, plated onto secondary plates, and incubated at 37° C. overnight.

Five colonies are picked, grown in 3 ml LB broth supplemented with kanamycin (100 μg/ml), and grown overnight at 37° C. Plasmid DNA is extracted using the Quiagen mini-prep method and is quantitated by agarose gel electrophoresis.

PCR is performed with the gene-specific primers under the conditions set forth above and transformant DNA is confirmed to contain the desired insert. If PCR-positive, one of the five plasmid DNA samples (500 ng) extracted from the E. coli XL1-blue cells is used to transform competent BL21 (λDE3) E. coli competent cells (Novagen; as described previously). Transformants (10) are picked, plated onto selective kanamycin (50 μg/ml)-containing LB agar plates, and stored as a research stock in LB containing 50% glycerol.

2.D. Purification of Recombinant Proteins

One ml of frozen glycerol stock prepared as described in 2.C. is used to inoculate 50 ml of LB medium containing 25 μg/ml kanamycin in a 250 ml Erlenmeyer flask. The flask is incubated at 37° C. for 2 hours or until the absorbance at 600 nm (OD₆₀₀) reaches 0.4-1.0. The culture is stopped from growing by placing the flask at 4° C. overnight. The following day, 10 ml of the overnight culture is used to inoculate 240 ml LB medium containing kanamycin (25 μg/ml), with the initial OD₆₀₀ being about 0.02-0.04. Four flasks are inoculated for each ORF. The cells are grown to an OD₆₀₀ of 1.0 (about 2 hours at 37° C.), a 1 ml sample is harvested by centrifugation, and the sample is analyzed by SDS-PAGE to detect any leaky expression. The remaining culture is induced with 1 mM IPTG and the induced cultures are grown for an additional 2 hours at 37° C.

The final OD₆₀₀ reading is taken and the cells are harvested by centrifugation at 5,000×g for 15 minutes at 4° C. The supernatant is discarded and the pellets are resuspended in 50 mM Tris-HCl (pH18.0), 2 mM EDTA. Two hundred and fifty ml of buffer are used for each 1 L of culture and the cells are recovered by centrifugation at 12,000×g for 20 minutes. The supernatant is discarded and the pellets are stored at −45° C.

2.E. Protein Purification

Pellets obtained using the methods described in 2.D. are thawed and resuspended in 95 ml of 50 mM Tris-HCl (pH 8.0). Pefabloc and lysozyme are added to final concentrations of 100 μM and 100 μg/ml, respectively. The mixture is homogenized with magnetic stirring at 5° C. for 30 minutes. Benzonase (Merck) is added to a final concentration of 1 U/ml, in the presence of 10 mM MgCl₂, to ensure total digestion of the DNA. The suspension is sonicated (Branson Sonifier 450) for 3 cycles of 2 minutes each at maximum output. The homogenate is centrifuged at 19,000×g for 15 minutes and both the supernatant and the pellet are analyzed by SDS-PAGE to detect the cellular location of the target protein in the soluble or insoluble fractions, as is described further below.

2.E.1. Soluble Fraction

If the target protein is produced in a soluble form (i.e., in the supernatant obtained using the methods described in 2.E.) NaCl and imidazole are added to the supernatant to final concentrations of 50 mM Tris-HCl (pH 8.0), 0.5 M NaCl, and 10 mM imidazole (buffer A). The mixture is filtered through a 0.45 μm membrane and loaded onto an IMAC column (Pharmacia HiTrap chelating Sepharose; 1 ml), which has been charged with nickel ions according to the manufacturer's recommendations. After loading, the column is washed with 50 column volumes of buffer A and the recombinant protein is eluted with 5 ml of buffer B (50 mM Tris-HCl (pH 8.0), 0.5 M NaCl, 500 mM imidazole).

The elution profile is monitored by measuring the absorbance of the fractions at 280 nm. Fractions corresponding to the protein peak are pooled, dialyzed against PBS containing 0.5 M arginine, filtered through a 0.22 μm membrane, and stored at −45° C.

2.E.2. Insoluble Fraction

If the target protein is expressed in the insoluble fraction (pellets obtained using the methods described in 2.E.), purification is conducted under denaturing conditions. NaCl, imidazole, and urea are added to the resuspended pellet to final concentrations of 50 mM Tris-HCl (pH 8.0), 0.5 M NaCl, 10 mM imidazole, and 6 M urea (buffer C). After complete solubilization, the mixture is filtered through a 0.45 μm membrane and loaded onto an IMAC column.

The purification procedures on the IMAC column are the same as are described in 2.E.1., except that 6 M urea is included in all of the buffers used and 10 column volumes of buffer C are used to wash the column after protein loading, instead of 50 column volumes.

The protein fractions eluted from the IMAC column with buffer D (buffer C containing 500 mM imidazole) are pooled. Arginine is added to the solution to a final concentration of 0.5 M, and the mixture is dialyzed against PBS containing 0.5 M arginine and various concentrations of urea (4 M, 3 M, 2 M, 1 M, and 0.5 M) to progressively decrease the concentration of urea. The final dialysate is filtered through a 0.22 μm membrane and stored at −45° C.

Alternatively, when the above-described purification process is not as efficient as it should be, two other processes can be used and are described as follows. A first alternative involves the use of a mild denaturant, N-octyl glucoside (NOG). Briefly, a pellet obtained as is described in 2.E. is homogenized in a solution of 5 mM imidazole, 500 mM sodium chloride, and 20 mM Tris-HCl (pH 7.9) by microfluidization at a pressure of 15,000 psi, and is clarified by centrifugation at 4,000-5,000×g. The pellet is recovered, resuspended in 50 mM NaPO₄ (pH 7.5) containing 1-2% weight/volume NOG, and homogenized. The NOG-soluble impurities are removed by centrifugation. The pellet is extracted once more by repeating the preceding extraction step. The pellet is dissolved in 8 M urea, 50 mM Tris (pH 8.0). The urea-solubilized protein is diluted with an equal volume of 2 M arginine, 50 mM Tris (pH 8.0), and is dialyzed against 1 M arginine for 24-48 hours to remove the urea. The final dialysate is filtered through a 0.22 μm membrane and stored at −45° C.

A second alternative involves the use of a strong denaturant, such as guanidine hydrochloride. Briefly, a pellet obtained as is described in 2.E. is homogenized in a solution of 5 mM imidazole, 500 mM sodium chloride, and 20 mM Tris-HCl (pH 7.9) by microfluidization at a pressure of 15,000 psi, and is clarified by centrifugation at 4,000-5,000×g. The pellet is recovered, resuspended in 6 M guanidine hydrochloride, and passed through an IMAC column charged with Ni⁺⁺. The bound antigen is eluted with 8 M urea (pH 8.5). β-mercaptoethanol is added to the eluted protein to a final concentration of 1 mM, and then the eluted protein is passed through a Sephadex G-25 column equilibrated in 0.1 M acetic acid. Protein eluted from the column is slowly added to 4 volumes of 50 mM phosphate buffer (H 7.0), and the protein remains in solution.

2.F. Evaluation of the Protective Activity of the Purified Protein

Groups of 10 OF1 mice (IFFA Credo) are immunized rectally with 25 μg of the purified recombinant protein, admixed with 1 μg of cholera toxin (Berna) in physiological buffer. Mice are immunized on days 0, 7, 14, and 21. Fourteen days after the last immunization, the mice are challenged with H. pylori strain ORV2001, grown in liquid media (the cells are grown on agar plates, as described in 2.A., and, after harvest, are resuspended in Brucella broth; the flasks are then incubated overnight at 37° C.). Fourteen days after challenge, the mice are sacrificed and their stomachs are removed. The amount of H. pylori is determined by measuring the urease activity in the stomach and by culture.

2.G. Production of Monospecific Polyclonal Antibodies

2.G.1. Hyperimmune Rabbit Antiserum

New Zealand rabbits are injected both subcutaneously and intramuscularly with 100 μg of a purified fusion polypeptide, as obtained using the methods described in 2.E.1. or 2.E.2., in the presence of Freund's complete adjuvant and in a total volume of approximately 2 ml. Twenty one and 42 days after the initial injection, booster doses, which are identical to the priming doses, except that Freund's incomplete adjuvant is used, are administered in the same way. Fifteen days after the last injection, animal serum is recovered, decomplemented, and filtered through a 0.45 μm membrane.

2.G.2. Mouse Hyperimmune Ascites Fluid

Ten mice are injected subcutaneously with 10-50 μg of a purified fusion polypeptide as obtained using the methods described in 2.E. 1. or 2.E.2., in the presence of Freund's complete adjuvant and in a volume of approximately 200 μl. Seven and 14 days after the initial injection, booster doses, which are identical to the priming doses, except that Freund's incomplete adjuvant is used, are administered in the same way. Twenty one and 28 days after the initial infection, mice receive 50 μg of the antigen alone intraperitoneally. On day 21, mice are also injected intraperitoneally with sarcoma 180/TG cells CM26684 (Lennette et al., Diagnostic Procedures for Viral, Rickettsial, and Chlamydial Infections, 5th Ed. Washington DC, American Public Health Association, 1979). Ascites fluid is collected 10-13 days after the last injection.

### EXAMPLE 3

### Methods for Producing Transcriptional Fusions Lacking His-tags

Methods for amplification and cloning of DNA encoding the polypeptides of the invention as transcriptional fusions lacking His-tags are described as follows. Two PCR primers for each clone are designed based upon the sequences of the polynucleotides that encode them (see the attached sequence listing, odd numbers, up to SEQ ID NO: 629). These primers can be used to amplify DNA encoding the polypeptides of the invention from any H. pylori strain, including, for example, ORV2001 and the strain deposited as ATCC deposit number 43579, as well as from other Helicobacter species.

The N-terminal primers are designed to include the ribosome binding site of the target gene, the ATG start site, and any signal sequence and cleavage site. The N-terminal primers can include a 5′ clamp and a restriction endonuclease recognition site, such as that for BamHI (GGATCC), which facilitates subsequent cloning. Similarly, the C-terminal primers can include a restriction endonuclease recognition site, such as that for XhoI (CTCGAG), which can be used in subsequent cloning, and a TAA stop codon.

Amplification of genes encoding the polypeptides of the invention can be carried out using Thermalase DNA Polymerase under the conditions described above in Example 2. Alternatively, Vent DNA polymerase (New England Biolabs), Pwo DNA polymerase (Boehringer Mannheim), or Taq DNA polymerase (Appligene) can be used, according to instructions provided by the manufacturers.

A single PCR product for each clone is amplified and cloned into appropriately cleaved pET 24 (e.g., BamHI-XhoI cleaved pET 24), resulting in the construction of a transcriptional fusion that permits expression of the proteins without His-tags. The expressed products can be purified as denatured proteins that are refolded by dialysis into 1 M arginine.

Cloning into pET 24 allows transcription of the genes from the T7 promoter, which is supplied by the vector, but relies upon binding of the RNA-specific DNA polymerase to the intrinsic ribosome binding sites of the genes, and thereby expression of the complete ORF. The amplification, digestion, and cloning protocols that can be used in this method are as described above for constructing translational fusions.

### EXAMPLE 4

### Purification of the Polypeptides of the Invention by Immunoaffinity

4.A. Purification of Specific IgGs

An immune serum, as prepared as is described in section 2.G., is applied to a protein A Sepharose Fast Flow column (Pharmacia) equilibrated in 100 mM Tris-HCl (pH 8.0). The resin is washed by applying 10 column volumes of 100 mM Tris-HCl and 10 volumes of 10 mM Tris-HCl (pH 8.0) to the column. IgG antibodies are eluted with 0.1 M glycine buffer (pH 3.0) and are collected as 5 ml fractions to each of which is added 0.25 ml 1 M Tris-HCl (pH 8.0). The optical density of the eluate is measured at 280 nm and fractions containing the IgG antibodies are pooled, dialyzed against 50 mM Tris-HCl (pH 8.0), and, if necessary, stored frozen at −70° C.

4.B. Preparation of the Column

An appropriate amount of CNBr-activated Sepharose 4B gel (1 g of dried gel provides for approximately 3.5 ml of hydrated gel; gel capacity is from 5 to 10 mg coupled IgG/ml of gel) manufactured by Pharmacia (17-0430-01) is suspended in 1 mM HCl buffer and washed with a buchner by adding small quantities of 1 mM HCl buffer. The total volume of buffer is 200 ml per gram of gel.

Purified IgG antibodies are dialyzed for 4 hours at 20±5° C. against 50 volumes of 500 mM sodium phosphate buffer (pH 7.5). The antibodies are then diluted in 500 mM phosphate buffer (pH 7.5) to a final concentration of 3 mg/ml.

IgG antibodies are mixed with the gel overnight at 5±3° C. The gel is packed into a chromatography column and is washed with 2 column volumes of 500 mM phosphate buffer (pH 7.5), and 1 column volume of 50 mM sodium phosphate buffer, containing 500 mM NaCl (pH 7.5). The gel is then transferred to a tube, mixed with 100 mM ethanolamine (pH 7.5) for 4 hours at room temperature, and washed twice with 2 column volumes of PBS. The gel is then stored in 1/10,000 PBS/merthiolate. The amount of IgG antibodies coupled to the gel is determined by measuring the optical density (OD) at 280 nm of the IgG solution and the direct eluate, plus washings.

4.C. Adsorption and Elution of the Antigen

An antigen solution in 50 mM Tris-HCl (pH 8.0), 2 mM EDTA, for example, the supernatant or the solubilized pellet obtained using the methods described in 3.E., after centrifugation and filtration through a 0.45 μm membrane, is applied to a column equilibrated with 50 mM Tris-HCl (pH 8.0), 2 mM EDTA, at a flow rate of about 10 ml/hour. The column is then washed with 20 volumes of 50 mM Tris-HCl (pH 8.0), 2 mM EDTA. Alternatively, adsorption can be achieved by mixing overnight at 5±3° C.

The adsorbed gel is washed with 2 to 6 volumes of 10 mM sodium phosphate buffer (pH 6.8) and the antigen is eluted with 100 mM glycine buffer (pH 2.5). The eluate is recovered in 3 ml fractions, to each of which is added 150 μl of 1 M sodium phosphate buffer (pH 8.0). Absorption is measured at 280 nm for each fraction; those fractions containing the antigen are pooled and stored at −20° C.

Other embodiments are within the following claims.

What is claimed is:

1. An isolated polynucleotide that encodes:
(i) a polypeptide comprising an amino acid sequence that is homologous to the amino acid sequence of a Helicobacter polypeptide selected from the group consisting of GHPO7 (SEQ ID NO: 2), GHPO8 (SEQ ID NO: 4), GHPO9 (SEQ ID NO: 6), GHPO10 (SEQ ID NO: 8), GHPO12 (SEQ ID NO: 10), GHPO25 (SEQ ID NO: 12), GHPO27 (SEQ ID NO: 14), GHPO29 (SEQ ID NO: 16), GHPO30 (SEQ ID NO: 18), GHPO37 (SEQ ID NO: 20), GHPO49 (SEQ ID NO: 22), GHPO51 (SEQ ID NO: 24), GHPO54 (SEQ ID NO: 26), GHPO65 (SEQ ID NO: 28), GHPO66 (SEQ ID NO: 30), GHPO68 (SEQ ID NO: 32), GHPO70 (SEQ ID NO: 34), GHPO77 (SEQ ID NO: 36), GHPO83 (SEQ ID NO: 38), GHPO85 (SEQ ID NO: 40), GHPO87 (SEQ ID NO: 42), GHPO91 (SEQ ID NO: 44), GHPO92 (SEQ ID NO: 46), GHPO96 (SEQ ID NO: 48), GHPO97 (SEQ ID NO: 50), GHPO111 (SEQ ID NO: 52), GHPO115 (SEQ ID NO: 54), GHPO117 (SEQ ID NO: 56), GHPO123 (SEQ ID NO: 58), GHPO124 (SEQ ID NO: 60), GHPO126 (SEQ ID NO: 62), GHPO127 (SEQ ID NO: 64), GHPO128 (SEQ ID NO: 66), GHPO131 (SEQ ID NO: 68), GHPO133 (SEQ ID NO: 70), GHPO140 (SEQ ID NO: 72), GHPO141 (SEQ ID NO: 74), GHPO145 (SEQ ID NO: 76), GHPO147 (SEQ ID NO: 78), GHPO166 (SEQ ID NO: 80), GHPO181 (SEQ ID NO: 82), GHPO187 (SEQ ID NO: 84), GHPO188 (SEQ ID NO: 86), GHPO192 (SEQ ID NO: 88), GHPO202 (SEQ ID NO: 90), GHPO204 (SEQ ID NO: 92), GHPO205 (SEQ ID NO: 94), GHPO212 (SEQ ID NO: 96), GHPO218 (SEQ ID NO: 98), GHPO226 (SEQ ID NO: 100), GHPO231 (SEQ ID NO: 102), GHPO236 (SEQ ID NO: 104), GHPO239 (SEQ ID NO: 106), GHPO245 (SEQ ID NO: 108), GHPO246 (SEQ ID NO: 110), GHPO248 (SEQ ID NO: 112), GHPO253 (SEQ ID NO: 114), GHPO265 (SEQ ID NO: 116), GHPO266 (SEQ ID NO: 118), GHPO271 (SEQ ID NO: 120), GHPO272 (SEQ ID NO: 122), GHPO286 (SEQ ID NO: 124), GHPO291 (SEQ ID NO: 126), GHPO292 (SEQ ID NO: 128), GHPO297 (SEQ ID NO: 130), GHPO304 (SEQ ID NO: 132), GHPO307 (SEQ ID NO: 134), GHPO324 (SEQ ID NO: 136), GHPO326 (SEQ ID NO: 138), GHPO331 (SEQ ID NO: 140), GHPO343 (SEQ ID NO: 142), GHPO345 (SEQ ID NO: 144), GHPO346 (SEQ ID NO: 146), GHPO352 (SEQ ID NO: 148), GHPO355 (SEQ ID NO: 150), GHPO363 (SEQ ID NO: 152), GHPO369 (SEQ ID NO: 154), GHPO376 (SEQ ID NO: 156), GHPO378 (SEQ ID NO: 158), GHPO388 (SEQ ID NO: 160), GHPO396 (SEQ ID NO: 162), GHPO403 (SEQ ID NO: 164), GHPO410 (SEQ ID NO: 166), GHPO415 (SEQ ID NO: 168), GHPO421 (SEQ ID NO: 170), GHPO439 (SEQ ID NO: 172), GHPO441 (SEQ ID NO: 174), GHPO443 (SEQ ID NO: 176), GHPO453 (SEQ ID NO: 178), GHPO455 (SEQ ID NO: 180), GHPO464 (SEQ ID NO: 182), GHPO467 (SEQ ID NO: 184), GHPO468 (SEQ ID NO: 186), GHPO470 (SEQ ID NO: 188), GHPO486 (SEQ ID NO: 190), GHPO487 (SEQ ID NO: 192), GHPO488 (SEQ ID NO: 194), GHPO489 (SEQ ID NO: 196), GHPO498 (SEQ ID NO: 198), GHPO501 (SEQ ID NO: 200), GHPO504 (SEQ ID NO: 202), GHPO512 (SEQ ID NO: 204), GHPO517 (SEQ ID NO: 206), GHPO520 (SEQ ID NO: 208), GHPO528 (SEQ ID NO: 210), GHPO530 (SEQ ID NO: 212), GHPO532 (SEQ ID NO: 214), GHPO548 (SEQ ID NO: 216), GHPO561 (SEQ ID NO: 218), GHPO564 (SEQ ID NO: 220), GHPO572 (SEQ ID NO: 222), GHPO573 (SEQ ID NO: 224), GHPO574 (SEQ ID NO: 226), GHPO577 (SEQ ID NO: 228), GHPO579 (SEQ ID NO: 230), GHPO583 (SEQ ID NO: 232), GHPO588 (SEQ ID NO: 234), GHPO593 (SEQ ID NO: 236), GHPO597 (SEQ ID NO: 238), GHPO598 (SEQ ID NO: 240), GHPO604 (SEQ ID NO: 242), GHPO606 (SEQ ID NO: 244), GHPO611 (SEQ ID NO: 246), GHPO612 (SEQ ID NO: 248), GHPO615 (SEQ ID NO: 250), GHPO632 (SEQ ID NO: 252), GHPO633 (SEQ ID NO: 254), GHPO637 (SEQ ID NO: 256), GHPO651 (SEQ ID NO: 258), GHPO663 (SEQ ID NO: 260), GHPO686 (SEQ ID NO: 262), GHPO693 (SEQ ID NO: 264), GHPO698 (SEQ ID NO: 266), GHPO703 (SEQ ID NO: 268), GHPO704 (SEQ ID NO: 270), GHPO705 (SEQ ID NO: 272), GHPO707 (SEQ ID NO: 274), GHPO721 (SEQ ID NO: 276), GHPO727 (SEQ ID NO: 278), GHPO728 (SEQ ID NO: 280), GHPO733 (SEQ ID NO: 282), GHPO758 (SEQ ID NO: 284), GHPO763 (SEQ ID NO: 286), GHPO771 (SEQ ID NO: 288), GHPO774 (SEQ ID NO: 290), GHPO776 (SEQ ID NO: 292), GHPO783 (SEQ ID NO: 294), GHPO800 (SEQ ID NO: 296), GHPO806 (SEQ ID NO: 298), GHPO807 (SEQ ID NO: 300), GHPO808 (SEQ ID NO: 302), GHPO809 (SEQ ID NO: 304), GHPO811 (SEQ ID NO: 306), GHPO815 (SEQ ID NO: 308), GHPO819 (SEQ ID NO: 310), GHPO841 (SEQ ID NO: 312), GHPO843 (SEQ ID NO: 314), GHPO846 (SEQ ID NO: 316), GHPO875 (SEQ ID NO: 318), GHPO892 (SEQ ID NO: 320), GHPO902 (SEQ ID NO: 322), GHPO904 (SEQ ID NO: 324), GHPO906 (SEQ ID NO: 326), GHPO908 (SEQ ID NO: 328), GHPO921 (SEQ ID NO: 330), GHPO923 (SEQ ID NO: 332), GHPO926 (SEQ ID NO: 334), GHPO933 (SEQ ID NO: 336), GHPO939 (SEQ ID NO: 338), GHPO940 (SEQ ID NO: 340), GHPO943 (SEQ ID NO: 342), GHPO951 (SEQ ID NO: 344), GHPO961 (SEQ ID NO: 346), GHPO965 (SEQ ID NO: 348), GHPO990 (SEQ ID NO: 350), GHPO991 (SEQ ID NO: 352), GHPO998 (SEQ ID NO: 354), GHPO1001 (SEQ ID NO: 356), GHPO1005 (SEQ ID NO: 358), GHPO1033 (SEQ ID NO: 360), GHPO1039 (SEQ ID NO: 362), GHPO1041 (SEQ ID NO: 364), GHPO1043 (SEQ ID NO: 366), GHPO1044 (SEQ ID NO: 368), GHPO1051 (SEQ ID NO: 370), GHPO1058 (SEQ ID NO: 372), GHPO1060 (SEQ ID NO: 374), GHPO1075 (SEQ ID NO: 376), GHPO1077 (SEQ ID NO: 378), GHPO1082 (SEQ ID NO: 380), GHPO1083 (SEQ ID NO: 382), GHPO1086 (SEQ ID NO: 384), GHPO1087 (SEQ ID NO: 386), GHPO1090 (SEQ ID NO: 388), GHPO1097 (SEQ ID NO: 390), GHPO1098 (SEQ ID NO: 392), GHPO1103 (SEQ ID NO: 394), GHPO1113 (SEQ ID NO: 396), GHPO1116 (SEQ ID NO: 398), GHPO1123 (SEQ ID NO: 400), GHPO1125 (SEQ ID NO: 402), GHPO1129 (SEQ ID NO: 404), GHPO1130 (SEQ ID NO: 406), GHPO1134 (SEQ ID NO: 408), GHPO1161 (SEQ ID NO: 410), GHPO1166 (SEQ ID NO: 412), GHPO1170 (SEQ ID NO: 414), GHPO1175 (SEQ ID NO: 416), GHPO1181 (SEQ ID NO: 418), GHPO1186 (SEQ ID NO: 420), GHPO1188 (SEQ ID NO: 422), GHPO1191 (SEQ ID NO: 424), GHPO1193 (SEQ ID NO: 426), GHPO1196 (SEQ ID NO: 428), GHPO1204 (SEQ ID NO: 430), GHPO1210 (SEQ ID NO: 432), GHPO1211 (SEQ ID NO: 434), GHPO1216 (SEQ ID NO: 436), GHPO1218 (SEQ ID NO: 438), GHPO1220 (SEQ ID NO: 440), GHPO1223 (SEQ ID NO: 442), GHPO1226 (SEQ ID NO: 444), GHPO1240 (SEQ ID NO: 446), GHPO1246 (SEQ ID NO: 448), GHPO1251 (SEQ ID NO: 450), GHPO1252 (SEQ ID NO: 452), GHPO1261 (SEQ ID NO: 454), GHPO1265 (SEQ ID NO: 456), GHPO1267 (SEQ ID NO: 458), GHPO1278 (SEQ ID NO: 460), GHPO1282 (SEQ ID NO: 462), GHPO1283 (SEQ ID NO: 464), GHPO1287 (SEQ ID NO: 466), GHPO1292 (SEQ ID NO: 468), GHPO1293 (SEQ ID NO: 470), GHPO1302 (SEQ ID NO: 472), GHPO1309 (SEQ ID NO: 474), GHPO1317 (SEQ ID NO: 476), GHPO1318 (SEQ ID NO: 478), GHPO1321 (SEQ ID NO: 480), GHPO1325 (SEQ ID NO: 482), GHPO1341 (SEQ ID NO: 484), GHPO1351 (SEQ ID NO: 486), GHPO1354 (SEQ ID NO: 488), GHPO1363 (SEQ ID NO: 490), GHPO1371 (SEQ ID NO: 492), GHPO1381 (SEQ ID NO: 494), GHPO1401 (SEQ ID NO: 496), GHPO1402 (SEQ ID NO: 498), GHPO1403 (SEQ ID NO: 500), GHPO1408 (SEQ ID NO: 502), GHPO1416 (SEQ ID NO: 504), GHPO1420 (SEQ ID NO: 506), GHPO1428 (SEQ ID NO: 508), GHPO1437 (SEQ ID NO: 510), GHPO1439 (SEQ ID NO: 512), GHPO1460 (SEQ ID NO: 514), GHPO1463 (SEQ ID NO: 516), GHPO1472 (SEQ ID NO: 518), GHPO1474 (SEQ ID NO: 520), GHPO1484 (SEQ ID NO: 522), GHPO1489 (SEQ ID NO: 524), GHPO1494 (SEQ ID NO: 526), GHPO1495 (SEQ ID NO: 528), GHPO1498 (SEQ ID NO: 530), GHPO1499 (SEQ ID NO: 532), GHPO1500 (SEQ ID NO: 534), GHPO1503 (SEQ ID NO: 536), GHPO1504 (SEQ ID NO: 538), GHPO1510 (SEQ ID NO: 540), GHPO1518 (SEQ ID NO: 542), GHPO1533 (SEQ ID NO: 544), GHPO1541 (SEQ ID NO: 546), GHPO1544 (SEQ ID NO: 548), GHPO1548 (SEQ ID NO: 550), GHPO1565 (SEQ ID NO: 552), GHPO1575 (SEQ ID NO: 554), GHPO1582 (SEQ ID NO: 556), GHPO1595 (SEQ ID NO: 558), GHPO1597 (SEQ ID NO: 560), GHPO1599 (SEQ ID NO: 562), GHPO1601 (SEQ ID NO: 564), GHPO1609 (SEQ ID NO: 566), GHPO1613 (SEQ ID NO: 568), GHPO1614 (SEQ ID NO: 570), GHPO1626 (SEQ ID NO: 572), GHPO1628 (SEQ ID NO: 574), GHPO1639 (SEQ ID NO: 576), GHPO1640 (SEQ ID NO: 578), GHPO1641 (SEQ ID NO: 580), GHPO1646 (SEQ ID NO: 582), GHPO1662 (SEQ ID NO: 584), GHPO1667 (SEQ ID NO: 586), GHPO1668 (SEQ ID NO: 588), GHPO1670 (SEQ ID NO: 590), GHPO1671 (SEQ ID NO: 592), GHPO1672 (SEQ ID NO: 594), GHPO1678 (SEQ ID NO: 596), GHPO1684 (SEQ ID NO: 598), GHPO1695 (SEQ ID NO: 600), GHPO1697 (SEQ ID NO: 602), GHPO1701 (SEQ ID NO: 604), GHPO1719 (SEQ ID NO: 606), GHPO1723 (SEQ ID NO: 608), GHPO1732 (SEQ ID NO: 610), GHPO1739 (SEQ ID NO: 612), GHPO1741 (SEQ ID NO: 614), GHPO1747 (SEQ ID NO: 616), GHPO1749 (SEQ ID NO: 618), GHPO1750 (SEQ ID NO: 620), GHPO1751 (SEQ ID NO: 622), GHPO1755 (SEQ ID NO: 624), GHPO1771 (SEQ ID NO: 626), GHPO1786 (SEQ ID NO: 628), and GHPO1789 (SEQ ID NO: 630); or
(ii) a derivative of said Helicobacter polypeptide.

2. The isolated polynucleotide of claim 1, which encodes a mature form of said Helicobacter polypeptide.

3. The isolated polynucleotide of claim 1, wherein the polynucleotide is a DNA molecule.

4. The isolated polynucleotide of claim 1, which is a DNA molecule that can be amplified by polymerase chain reaction from a Helicobacter genome.

5. The isolated DNA molecule of claim 4, which can be amplified by the polymerase chain reaction from a Helicobacter pylori genome.

6. The isolated polynucleotide of claim 1, which is a DNA molecule that encodes the mature form or a derivative of a polypeptide encoded by the DNA molecule of claim 4.

7. The isolated polynucleotide of claim 1, which is a DNA molecule that encodes the mature form or a derivative of a polypeptide encoded by the DNA molecule of claim 5.

8. A compound, in a substantially purified form, that is the mature form or a derivative of a polypeptide comprising an amino acid sequence that is homologous to a Helicobacter polypeptide selected from the group consisting of GHPO7 (SEQ ID NO: 2), GHPO8 (SEQ ID NO: 4), GHPO9 (SEQ ID NO: 6), GHPO10 (SEQ ID NO: 8), GHPO12 (SEQ ID NO: 10), GHPO25 (SEQ ID NO: 12), GHPO27 (SEQ ID NO: 14), GHPO29 (SEQ ID NO: 16), GHPO30 (SEQ ID NO: 18), GHPO37 (SEQ ID NO: 20), GHPO49 (SEQ ID NO: 22), GHPO51 (SEQ ID NO: 24), GHPO54 (SEQ ID NO: 26), GHPO65 (SEQ ID NO: 28), GHPO66 (SEQ ID NO: 30), GHPO68 (SEQ ID NO: 32), GHPO70 (SEQ ID NO: 34), GHPO77 (SEQ ID NO: 36), GHPO83 (SEQ ID NO: 38), GHPO85 (SEQ ID NO: 40), GHPO87 (SEQ ID NO: 42), GHPO91 (SEQ ID NO: 44), GHPO92 (SEQ ID NO: 46), GHPO96 (SEQ ID NO: 48), GHPO97 (SEQ ID NO: 50), GHPO111 (SEQ ID NO: 52), GHPO115 (SEQ ID NO: 54), GHPO117 (SEQ ID NO: 56), GHPO123 (SEQ ID NO: 58), GHPO124 (SEQ ID NO: 60), GHPO126 (SEQ ID NO: 62), GHPO127 (SEQ ID NO: 64), GHPO128 (SEQ ID NO: 66), GHPO131 (SEQ ID NO: 68), GHPO133 (SEQ ID NO: 70), GHPO140 (SEQ ID NO: 72), GHPO141 (SEQ ID NO: 74), GHPO145 (SEQ ID NO: 76), GHPO147 (SEQ ID NO: 78), GHPO166 (SEQ ID NO: 80), GHPO181 (SEQ ID NO: 82), GHPO187 (SEQ ID NO: 84), GHPO188 (SEQ ID NO: 86), GHPO192 (SEQ ID NO: 88), GHPO202 (SEQ ID NO: 90), GHPO204 (SEQ ID NO: 92), GHPO205 (SEQ ID NO: 94), GHPO212 (SEQ ID NO: 96), GHPO218 (SEQ ID NO: 98), GHPO226 (SEQ ID NO: 100), GHPO231 (SEQ ID NO: 102), GHPO236 (SEQ ID NO: 104), GHPO239 (SEQ ID NO: 106), GHPO245 (SEQ ID NO: 108), GHPO246 (SEQ ID NO: 110), GHPO248 (SEQ ID NO: 112), GHPO253 (SEQ ID NO: 114), GHPO265 (SEQ ID NO: 116), GHPO266 (SEQ ID NO: 118), GHPO271 (SEQ ID NO: 120), GHPO272 (SEQ ID NO: 122), GHPO286 (SEQ ID NO: 124), GHPO291 (SEQ ID NO: 126), GHPO292 (SEQ ID NO: 128), GHPO297 (SEQ ID NO: 130), GHPO304 (SEQ ID NO: 132), GHPO307 (SEQ ID NO: 134), GHPO324 (SEQ ID NO: 136), GHPO326 (SEQ ID NO: 138), GHPO331 (SEQ ID NO: 140), GHPO343 (SEQ ID NO: 142), GHPO345 (SEQ ID NO: 144), GHPO346 (SEQ ID NO: 146), GHPO352 (SEQ ID NO: 148), GHPO355 (SEQ ID NO: 150), GHPO363 (SEQ ID NO: 152), GHPO369 (SEQ ID NO: 154), GHPO376 (SEQ ID NO: 156), GHPO378 (SEQ ID NO: 158), GHPO388 (SEQ ID NO: 160), GHPO396 (SEQ ID NO: 162), GHPO403 (SEQ ID NO: 164), GHPO410 (SEQ ID NO: 166), GHPO415 (SEQ ID NO: 168), GHPO421 (SEQ ID NO: 170), GHPO439 (SEQ ID NO: 172), GHPO441 (SEQ ID NO: 174), GHPO443 (SEQ ID NO: 176), GHPO453 (SEQ ID NO: 178), GHPO455 (SEQ ID NO: 180), GHPO464 (SEQ ID NO: 182), GHPO467 (SEQ ID NO: 184), GHPO468 (SEQ ID NO: 186), GHPO470 (SEQ ID NO: 188), GHPO486 (SEQ ID NO: 190), GHPO487 (SEQ ID NO: 192), GHPO488 (SEQ ID NO: 194), GHPO489 (SEQ ID NO: 196), GHPO498 (SEQ ID NO: 198), GHPO501 (SEQ ID NO: 200), GHPO504 (SEQ ID NO: 202), GHPO512 (SEQ ID NO: 204), GHPO517 (SEQ ID NO: 206), GHPO520 (SEQ ID NO: 208), GHPO528 (SEQ ID NO: 210), GHPO530 (SEQ ID NO: 212), GHPO532 (SEQ ID NO: 214), GHPO548 (SEQ ID NO: 216), GHPO561 (SEQ ID NO: 218), GHPO564 (SEQ ID NO: 220), GHPO572 (SEQ ID NO: 222), GHPO573 (SEQ ID NO: 224), GHPO574 (SEQ ID NO: 226), GHPO577 (SEQ ID NO: 228), GHPO579 (SEQ ID NO: 230), GHPO583 (SEQ ID NO: 232), GHPO588 (SEQ ID NO: 234), GHPO593 (SEQ ID NO: 236), GHPO597 (SEQ ID NO: 238), GHPO598 (SEQ ID NO: 240), GHPO604 (SEQ ID NO: 242), GHPO606 (SEQ ID NO: 244), GHPO611 (SEQ ID NO: 246), GHPO612 (SEQ ID NO: 248), GHPO615 (SEQ ID NO: 250), GHPO632 (SEQ ID NO: 252), GHPO633 (SEQ ID NO: 254), GHPO637 (SEQ ID NO: 256), GHPO651 (SEQ ID NO: 258), GHPO663 (SEQ ID NO: 260), GHPO686 (SEQ ID NO: 262), GHPO693 (SEQ ID NO: 264), GHPO698 (SEQ ID NO: 266), GHPO703 (SEQ ID NO: 268), GHPO704 (SEQ ID NO: 270), GHPO705 (SEQ ID NO: 272), GHPO707 (SEQ ID NO: 274), GHPO721 (SEQ ID NO: 276), GHPO727 (SEQ ID NO: 278), GHPO728 (SEQ ID NO: 280), GHPO733 (SEQ ID NO: 282), GHPO758 (SEQ ID NO: 284), GHPO763 (SEQ ID NO: 286), GHPO771 (SEQ ID NO: 288), GHPO774 (SEQ ID NO: 290), GHPO776 (SEQ ID NO: 292), GHPO783 (SEQ ID NO: 294), GHPO800 (SEQ ID NO: 296), GHPO806 (SEQ ID NO: 298), GHPO807 (SEQ ID NO: 300), GHPO808 (SEQ ID NO: 302), GHPO809 (SEQ ID NO: 304), GHPO811 (SEQ ID NO: 306), GHPO815 (SEQ ID NO: 308), GHPO819 (SEQ ID NO: 310), GHPO841 (SEQ ID NO: 312), GHPO843 (SEQ ID NO: 314), GHPO846 (SEQ ID NO: 316), GHPO875 (SEQ ID NO: 318), GHPO892 (SEQ ID NO: 320), GHPO902 (SEQ ID NO: 322), GHPO904 (SEQ ID NO: 324), GHPO906 (SEQ ID NO: 326), GHPO908 (SEQ ID NO: 328), GHPO921 (SEQ ID NO: 330), GHPO923 (SEQ ID NO: 332), GHPO926 (SEQ ID NO: 334), GHPO933 (SEQ ID NO: 336), GHPO939 (SEQ ID NO: 338), GHPO940 (SEQ ID NO: 340), GHPO943 (SEQ ID NO: 342), GHPO951 (SEQ ID NO: 344), GHPO961 (SEQ ID NO: 346), GHPO965 (SEQ ID NO: 348), GHPO990 (SEQ ID NO: 350), GHPO991 (SEQ ID NO: 352), GHPO998 (SEQ ID NO: 354), GHPO1001 (SEQ ID NO: 356), GHPO1005 (SEQ ID NO: 358), GHPO1033 (SEQ ID NO: 360), GHPO1039 (SEQ ID NO: 362), GHPO1041 (SEQ ID NO: 364), GHPO1043 (SEQ ID NO: 366), GHPO1044 (SEQ ID NO: 368), GHPO1051 (SEQ ID NO: 370), GHPO1058 (SEQ ID NO: 372), GHPO1060 (SEQ ID NO: 374), GHPO1075 (SEQ ID NO: 376), GHPO1077 (SEQ ID NO: 378), GHPO1082 (SEQ ID NO: 380), GHPO1083 (SEQ ID NO: 382), GHPO1086 (SEQ ID NO: 384), GHPO1087 (SEQ ID NO: 386), GHPO1090 (SEQ ID NO: 388), GHPO1097 (SEQ ID NO: 390), GHPO1098 (SEQ ID NO: 392), GHPO1103 (SEQ ID NO: 394), GHPO1113 (SEQ ID NO: 396), GHPO1116 (SEQ ID NO: 398), GHPO1123 (SEQ ID NO: 400), GHPO1125 (SEQ ID NO: 402), GHPO1129 (SEQ ID NO: 404), GHPO1130 (SEQ ID NO: 406), GHPO1134 (SEQ ID NO: 408), GHPO1161 (SEQ ID NO: 410), GHPO1166 (SEQ ID NO: 412), GHPO1170 (SEQ ID NO: 414), GHPO1175 (SEQ ID NO: 416), GHPO1181 (SEQ ID NO: 418), GHPO1186 (SEQ ID NO: 420), GHPO1188 (SEQ ID NO: 422), GHPO1191 (SEQ ID NO: 424), GHPO1193 (SEQ ID NO: 426), GHPO1196 (SEQ ID NO: 428), GHPO1204 (SEQ ID NO: 430), GHPO1210 (SEQ ID NO: 432), GHPO1211 (SEQ ID NO: 434), GHPO1216 (SEQ ID NO: 436), GHPO1218 (SEQ ID NO: 438), GHPO1220 (SEQ ID NO: 440), GHPO1223 (SEQ ID NO: 442), GHPO1226 (SEQ ID NO: 444), GHPO1240 (SEQ ID NO: 446), GHPO1246 (SEQ ID NO: 448), GHPO1251 (SEQ ID NO: 450), GHPO1252 (SEQ ID NO: 452), GHPO1261 (SEQ ID NO: 454), GHPO1265 (SEQ ID NO: 456), GHPO1267 (SEQ ID NO: 458), GHPO1278 (SEQ ID NO: 460), GHPO1282 (SEQ ID NO: 462), GHPO1283 (SEQ ID NO: 464), GHPO1287 (SEQ ID NO: 466), GHPO1292 (SEQ ID NO: 468), GHPO1293 (SEQ ID NO: 470), GHPO1302 (SEQ ID NO: 472), GHPO1309 (SEQ ID NO: 474), GHPO1317 (SEQ ID NO: 476), GHPO1318 (SEQ ID NO: 478), GHPO1321 (SEQ ID NO: 480), GHPO1325 (SEQ ID NO: 482), GHPO1341 (SEQ ID NO: 484), GHPO1351 (SEQ ID NO: 486), GHPO1354 (SEQ ID NO: 488), GHPO1363 (SEQ ID NO: 490), GHPO1371 (SEQ ID NO: 492), GHPO1381 (SEQ ID NO: 494), GHPO1401 (SEQ ID NO: 496), GHPO1402 (SEQ ID NO: 498), GHPO1403 (SEQ ID NO: 500), GHPO1408 (SEQ ID NO: 502), GHPO1416 (SEQ ID NO: 504), GHPO1420 (SEQ ID NO: 506), GHPO1428 (SEQ ID NO: 508), GHPO1437 (SEQ ID NO: 510), GHPO1439 (SEQ ID NO: 512), GHPO1460 (SEQ ID NO: 514), GHPO1463 (SEQ ID NO: 516), GHPO1472 (SEQ ID NO: 518), GHPO1474 (SEQ ID NO: 520), GHPO1484 (SEQ ID NO: 522), GHPO1489 (SEQ ID NO: 524), GHPO1494 (SEQ ID NO: 526), GHPO1495 (SEQ ID NO: 528), GHPO1498 (SEQ ID NO: 530), GHPO1499 (SEQ ID NO: 532), GHPO1500 (SEQ ID NO: 534), GHPO1503 (SEQ ID NO: 536), GHPO1504 (SEQ ID NO: 538), GHPO1510 (SEQ ID NO: 540), GHPO1518 (SEQ ID NO: 542), GHPO1533 (SEQ ID NO: 544), GHPO1541 (SEQ ID NO: 546), GHPO1544 (SEQ ID NO: 548), GHPO1548 (SEQ ID NO: 550), GHPO1565 (SEQ ID NO: 552), GHPO1575 (SEQ ID NO: 554), GHPO1582 (SEQ ID NO: 556), GHPO1595 (SEQ ID NO: 558), GHPO1597 (SEQ ID NO: 560), GHPO1599 (SEQ ID NO: 562), GHPO1601 (SEQ ID NO: 564), GHPO1609 (SEQ ID NO: 566), GHPO1613 (SEQ ID NO: 568), GHPO1614 (SEQ ID NO: 570), GHPO1626 (SEQ ID NO: 572), GHPO1628 (SEQ ID NO: 574), GHPO1639 (SEQ ID NO: 576), GHPO1640 (SEQ ID NO: 578), GHPO1641 (SEQ ID NO: 580), GHPO1646 (SEQ ID NO: 582), GHPO1662 (SEQ ID NO: 584), GHPO1667 (SEQ ID NO: 586), GHPO1668 (SEQ ID NO: 588), GHPO1670 (SEQ ID NO: 590), GHPO1671 (SEQ ID NO: 592), GHPO1672 (SEQ ID NO: 594), GHPO1678 (SEQ ID NO: 596), GHPO1684 (SEQ ID NO: 598), GHPO1695 (SEQ ID NO: 600), GHPO1697 (SEQ ID NO: 602), GHPO1701 (SEQ ID NO: 604), GHPO1719 (SEQ ID NO: 606), GHPO1723 (SEQ ID NO: 608), GHPO1732 (SEQ ID NO: 610), GHPO1739 (SEQ ID NO: 612), GHPO1741 (SEQ ID NO: 614), GHPO1747 (SEQ ID NO: 616), GHPO1749 (SEQ ID NO: 618), GHPO1750 (SEQ ID NO: 620), GHPO1751 (SEQ ID NO: 622), GHPO1755 (SEQ ID NO: 624), GHPO1771 (SEQ ID NO: 626), GHPO1786 (SEQ ID NO: 628), and GHPO1789 (SEQ ID NO: 630); or
(ii) a derivative of said Helicobacter polypeptide.

9. The compound of claim 8, which is the mature form or a derivative of a polypeptide encoded by a DNA molecule of claim 4.

10. The compound of claim 8, which is the mature form or a derivative of a polypeptide encoded by a DNA molecule of claim 5.

11. A method of preventing or treating Helicobacter infection in a mammal, said method comprising administering to said mammal a prophylactically or therapeutically effective amount of a compound of claim 8.

12. The method of claim 11, further comprising administering to said mammal an antibiotic, an antisecretory agent, a bismuth salt, or a combination thereof.

13. The method of claim 12, wherein said antibiotic is selected from the group consisting of amoxicillin, clarithromycin, tetracycline, metronidizole, and erythromycin.

14. The method of claim 12, wherein said bismuth salt is selected from the group consisting of bismuth subcitrate and bismuth subsalicylate.

15. The method of claim 12, wherein said antisecretory agent is a proton pump inhibitor.

16. The method of claim 15, wherein said proton pump inhibitor is selected from the group consisting of omeprazole, lansoprazole, and pantoprazole.

17. The method of claim 12, wherein said antisecretory agent is an H₂-receptor antagonist.

18. The method of claim 17, wherein said H₂-receptor antagonist is selected from the group consisting of ranitidine, cimetidine, famotidine, nizatidine, and roxatidine.

19. The method of claim 12, wherein said antisecretory agent is a prostaglandin analog.

20. The method of claim 19, wherein said prostaglandin analog is misoprostil or enprostil.

21. The method of claim 11, further comprising administering to said mammal a prophylactically or therapeutically effective amount of a second Helicobacter polypeptide or a derivative thereof.

22. The method of claim 21, wherein the second Helicobacter polypeptide is a Helicobacter urease, or a subunit or a derivative thereof.

23. A composition comprising a compound of claim 8, together with a physiologically acceptable diluent or carrier.

24. The composition of claim 23, further comprising an adjuvant.

25. The composition of claim 23, further comprising a second Helicobacter polypeptide or a derivative thereof.

26. The composition of claim 25, wherein said second Helicobacter polypeptide is a Helicobacter urease, or a subunit or a derivative thereof.

27. A method of preventing or treating Helicobacter infection in a mammal, said method comprising administering to said mammal a prophylactically or therapeutically effective amount of a polynucleotide of claim 1.

28. A method of preventing or treating Helicobacter infection in a mammal, said method comprising administering to said mammal a prophylactically or therapeutically effective amount of a polynucleotide of claim 4.

29. A method of preventing or treating Helicobacter infection in a mammal, said method comprising administering to said mammal a prophylactically or therapeutically effective amount of a polynucleotide of claim 7.

30. A composition comprising a viral vector, in the genome of which is inserted a DNA molecule of claim 3, said DNA molecule being placed under conditions for expression in a mammalian cell and said viral vector being admixed with a physiologically acceptable diluent or carrier.

31. The composition of claim 30, wherein said viral vector is a poxvirus.

32. A composition that comprises a bacterial vector comprising a DNA molecule of claim 3, said DNA molecule being placed under conditions for expression and said bacterial vector being admixed with a physiologically acceptable diluent or carrier.

33. The composition of claim 32, wherein said vector is selected from the group consisting of Shigella, Salmonella, Vibrio cholerae, Lactobacillus, Bacille bilié de Calmette-Guérin, and Streptococcus.

34. A composition comprising a polynucleotide of claim 1, together with a physiologically acceptable diluent or carrier.

35. The composition of claim 34, wherein said polynucleotide is a DNA molecule that is inserted in a plasmid that is unable to replicate and to substantially integrate in a mammalian genome and is placed under conditions for expression in a mammalian cell.

36. An expression cassette comprising a DNA molecule of claim 3, said DNA molecule being placed under conditions for expression in a procaryotic or eucaryotic cell.

37. A process for producing a compound of claim 8, which comprises culturing a procaryotic or eucaryotic cell transformed or transfected with an expression cassette of claim 36, and recovering said compound from the cell culture.

38. A method of preventing or treating Helicobacter infection in a mammal, said method comprising administering to said mammal a prophylactically or therapeutically effective amount of an antibody that binds to the compound of claim 8.
